(19) United States
(12) Patent Application Publication
(10) Pub. No.: US 2023/0330053 A1
(43) Pub. Date: Oct. 19, 2023

(54) METHOD FOR TREATING INFECTIONS

(71) Applicant: EcoPlanet Environmental LLC, Belgrade, MT (US)

(72) Inventors: Gary A. STROBEL, Bozeman, MT (US); Bryan BLATT, Manhattan, MT (US)

(21) Appl. No.: 18/339,206

(22) Filed: Jun. 21, 2023

Related U.S. Application Data
(62) Division of application No. 17/366,668, filed on Jul. 2, 2021.
(60) Provisional application No. 63/141,271, filed on Jan. 25, 2021.
(60) Provisional application No. 63/123,069, filed on Dec. 9, 2020.
(60) Provisional application No. 63/110,151, filed on Nov. 5, 2020.
(60) Provisional application No. 63/090,913, filed on Oct. 13, 2020.
(60) Provisional application No. 63/059,584, filed on Jul. 31, 2020.
(60) Provisional application No. 63/047,672, filed on Jul. 2, 2020.

(51) Int. Cl. A61K 31/22; A61K 31/19; A61L 2/00; A61L 11/00; A23K 20/105

(52) U.S. Cl. CPC A61K 31/22; A61K 31/19; A61L 2/0082; A61L 11/00; A23K 20/105

(57) ABSTRACT

The composition for treating infections may be used for treating a wide variety of different infection and conditions, including viral infections, such as coronavirus disease 2019 (COVID-19) and influenza. The composition includes isoamyl hexanoates and at least one acid. The at least one acid may be lactic acid, propanoic acid, isobutyric acid, butyric acid, lactic acid, formic acid, acetic acid, citric acid, oxalic acid, uric acid, malic acid, tartaric acid, or combinations thereof. The composition may be provided in any suitable form, including, but not limited to, a cream, an ointment, a rinse, an oil, a scrub, a spray, a shampoo, a gel, a plaster, a paste, a solution, a suspension, a dip, a salve, an ear rinse, a powder, an eyewash, mouthwash, a nail lacquer, a gas or an orally administered treatment. The composition may also be used as a feed or feed supplement, or a waste treatment.

### CROSS-REFERENCE TO RELATED APPLICATION

This application is a divisional of U.S. patent application Ser. No. 17/366,668, filed on Jul. 2, 2021, which claims the benefit of U.S. Provisional Patent Application No. 63/141,271, filed on Jan. 25, 2021; U.S. Provisional Patent Application No. 63/123,069, filed on Dec. 9, 2020; U.S. Provisional Patent Application No. 63/110,151, filed on Nov. 5, 2020; U.S. Provisional Patent Application No. 63/090,913 filed on Oct. 13, 2020; U.S. Provisional Patent Application No. 63/059,584 filed on Jul. 31, 2020; and U.S. Provisional Patent Application No. 63/047,672, filed on Jul. 2, 2020.

### BACKGROUND

### 1. Field

The disclosure of the present patent application relates to the treatment of infections, including bacterial, fungal, parasitic, protozoan, and viral infections, and particularly to a composition including isoamyl hexanoates and at least one acid, such as lactic acid, propanoic acid, isobutyric acid, butyric acid, lactic acid, formic acid, acetic acid, citric acid, oxalic acid, uric acid, malic acid, tartaric acid or combinations thereof. Further, the composition has shown effectiveness for the treatment of coronavirus disease 2019 (COVID-19) (Human USA-WA 1/2020 strain) and influenza (2009 pandemic H1N1 strain containing the H275Y NA mutation).

The composition may be incorporated into a human or animal food or food supplement, and particularly may be used in the form of, for example, an animal feed composition, to provide a supplement for treating infections, including bacterial, fungal, parasitic, protozoan and viral infections, and that improves gut health, may be used for weight gain, may be used as an immune modulator, and may further be used as a feed preservative.

The composition may also be in a form useful for treating animal or human waste, including, but not limited to, decontaminating, degrading, and/or deodorizing the waste. The composition could be administered directly to waste, or, for example, to litter (such as cat litter), or to bedding of the animal or human.

### 2. Description of the Related Art

Due to the ability of microorganisms to evolve and adapt to antimicrobial agents, there is a constant need for new treatments for infections of all types. There is a particular need for broad spectrum treatments which can be used to treat a wide variety of conditions. Unfortunately, such broad spectrum treatments are not only rare, but they are also very difficult and expensive to manufacture. Further, such treatments are typically limited in how they may be administered. For example, an oral treatment may not be able to be adapted into a topical treatment, or a topical treatment may not be able to be adapted for oral usage.

It would be desirable to be able to provide a single broad-spectrum agent which can be adapted to application in a wide variety of different ways, including adaptation for oral usage in humans or animals. These compositions and uses for treating infections could be useful not only for direct treatment or prevention of specific conditions in humans or animals, but also as human or animal food or food supplements, and compositions for treating human or animal waste in an efficient, environmentally friendly, and cost-effective manner.

A novel endophytic fungus, Muscodor albus, was isolated from Cinnamomum zeylanicum and shown to possess the rare capability of having wide antimicrobial activity mainly associated with its gas phase. This organism, in pure culture, produced more than two dozen volatile organic compounds (VOCs) and these were demonstrated, in half moon agar Petri plate assays, to be responsible for the bioactivity of this endophyte. That the VOCs were responsible for the antimicrobial activity was further demonstrated by acquiring the majority of the individual VOCs, placing them in a mixture in the same relative molar ratio that they were detected by gas chromatography/mass spectroscopy (GC/MS), and then testing their activity against a range of pathogenic fungi and bacteria. Interestingly, the artificial mixture virtually mimicked the activity of the fungus itself. Subsequently, individual compounds, combinations of two compounds and three compounds were tested against a range of target microorganisms in order to learn which were necessary to provide the greatest antimicrobial activity. It was demonstrated that central to the bioactivity of the VOCs, and usually produced in the greatest amount in culture, was a small molecular weight organic acid, isobutyric acid, which, by itself, possessed some limited bioactivity both in gas and minimum inhibitory concentration (MIC) assays. However, when placed with one or more complex esters, having little or no activity depending upon the target organism, the mixture was greatly increased in its antimicrobial activity and the concept of synergism could be used to define the effect; i.e., the activity observed was greater than that of either the acid or the esters alone.

Ultimately, it was learned that a mixture of a particular acid and a specific ester placed in an electrolyte solution was extremely effective in treating calves and other animals suffering with diarrhea (i.e., “scours”). The acid in this case was propionic acid and/or isobutyric acid, along with isoamyl hexanoates, having been substituted for any number of other esters made by Muscodor sp, and this was designated “Sx”. These substitutions were inspired by the bioactive formulae of the Muscodor sp., however, the Sx formulation is not identical to any Muscodor components. It was also observed that Sx was biologically active against a wide range of both human and plant pathogenic fungi and bacteria, including such microbes as Listeria sp., Salmonella sp., drug resistant Staphyloccus aureus, Clostridium sp., Xanthomonas sp. Candida albicans, Pythium ultimum, and Rhizoctonia solani. Thus, while the Sx had been demonstrated to be active against both bacteria and fungi, there had been no indication that Sx would inactivate any virus until the Porcine Epidemic Diarrhea (PED) virus outbreak in pigs in the United States in 2013-2014.

In Montana, one grower lost 850 piglets within a few weeks once the virus reached his locale. The disease was confirmed by Newport Labs of Worthington, MN. The Sx electrolyte solution was administered to tens of animals suffering with PED symptoms. Many of the treated animals seemed better within 5 hours, and all were completely recovered within 24 hours. Post infection assays revealed that no PED virus was lingering in any of the animals that were treated. The PED virus is a coronavirus, thus investigation of whether or not Sx could be used to treat SARS-CoV-2 is of great interest. It is estimated that 20% of COVID-19 patients suffer from severe diarrhea, thus there is potential for treatment of COVID-19 using Sx, as it has proven effective generally against a similar coronavirus, and specifically against a similar condition.

Of additional concern is acute gastroenteritis, which is a diarrheal disease with rapid onset, potentially accompanied by nausea, vomiting, fever or abdominal pain. Diarrhea and other gastrointestinal symptoms often cause dehydration, which can be especially dangerous in infants and small children. Acute gastroenteritis is a common infection impacting both developed and developing nations. Acute gastroenteritis may be caused by a variety of pathogens, including bacteria, viruses, and select parasites. Viruses are widely regarded as the type of pathogen most frequently involved in acute gastroenteritis. Viruses commonly implicated in acute gastroenteritis include norovirus, rotavirus, astrovirus, and adenovirus strains. Norovirus infections are most frequently associated with viral gastroenteritis.

In addition to viral causes of acute gastroenteritis, bacteria may also be responsible for infection. While several bacteria strains may cause acute gastroenteritis, campylobacter, Salmonella, and Shigella are most commonly involved in the condition. Lastly, certain parasitic species, including giardia and Cryptosporidium, may cause acute gastroenteritis.

Given the relatively mild symptomology of acute gastroenteritis, treatment is often limited to addressing symptoms and replenishing fluids. However, prevention of acute gastroenteritis is of large concern, particularly among infants and young children where infection may be particularly dangerous. To date, two rotavirus vaccines have been approved to treat infants, namely, RotaTeq, manufactured by Merck, was approved in 2006 for prevention of rotavirus gastroenteritis caused by types G1, G2, G3 and G4. RotaTeq is given as a three-time dosing regiment at 2, 4 and 6 months of age. Rotarix, manufactured by GSK, is also available. Approved in 2008, Rotarix is indicated to prevent rotavirus gastroenteritis caused by G1, G3, G4 and G9 types, with doses given at 2 and 4 months of age. Efficacy for both vaccines is estimated at 60-90%. No vaccines have been approved for norovirus, though there are several vaccine candidates under consideration.

In addition to the risk of severe symptoms or death, acute gastroenteritis also has a substantial economic impact. In the U.S., acute gastroenteritis is estimated to cost at least $250 million in direct medical costs, with more than $1 billion total cost to society. Direct costs are likely due, primarily, to hospitalization costs for young children, while indirect costs are driven by lost productivity.

On a global scale, the direct and indirect costs are more difficult to measure, as per patient costs may vary widely by country. However, norovirus alone is estimated to cause $4.2 billion in direct health system costs and $60.3 billion in societal costs. Given the economic burden of norovirus, the most common cause of acute gastroenteritis as a reference point, it is clear that the global cost of acute gastroenteritis is significant.

In addition to pathogens listed previously, coronaviruses have been known to cause acute gastroenteritis. An underrecognized hallmark of SARS-CoV-2 are gastrointestinal symptoms that may include acute gastroenteritis. Unfortunately, the availability of a SARS-COV-2 vaccine is unlikely to immediately remedy the worldwide pandemic. Thus, it remains imperative to have effective treatments for SARS-CoV-2. Thus, a composition and method for treating infections solving the aforementioned problems are desired.

### SUMMARY

The composition for treating infections may be used for treating a wide variety of different infection and conditions, including bacterial, parasitic, protozoan, fungal and viral infections. The composition includes isoamyl hexanoates and at least one acid. The at least one acid may be lactic acid, propanoic acid, isobutyric acid, butyric acid, lactic acid, formic acid, acetic acid, citric acid, oxalic acid, uric acid, malic acid, tartaric acid or combinations thereof. The composition may be provided in any suitable form, including, but not limited to, a cream, an ointment, a rinse, an oil, a scrub, a spray, a shampoo, a gel, a plaster, a solution, a suspension, a dip, a salve, an ear rinse, a powder, an eyewash, mouthwash, a nail lacquer, a gas or gas phase, an oral electrolyte solution or an orally administered treatment. The composition may also be used as a treatment for wound care; as a food or food supplement; or as a treatment of human or animal waste.

In use as a direct treatment or prevention of an infection, an effective amount of the composition is administered to a patient suffering from a condition caused by an infective agent, or as a preventative of such a condition. The patient may be a human being or an animal. Non-limiting examples of conditions to be treated with the composition include coronavirus disease 2019 (COVID-19), influenza, and other viral infections. Additionally, the composition may be used as a treatment for biosecurity decontamination and in the processing of meat, poultry, eggs and other foodstuffs, particularly with regard to viral contaminants. In addition to the above examples, the composition may be integrated into animal feed or an animal feed supplement for an animal patient. Non-limiting examples of conditions to be treated with the composition include a fungal infection, infection with protozoa, mastitis, rain rot, scald, foot rot, an ear infection, ring worm, dermatitis, a bacterial infection, a parasitic infection, a viral infection, diarrhea, a Streptomyces avermitilis infection, vaginitis, an oral infection, a nasal infection, a throat infection, jock itch, vaginosis, a toenail infection, a fingernail infection, an eye infection, and combinations thereof.

As an animal feed composition, the formulation may be prepared as a single- or multi-species animal feed made from a base animal feed, a carrier, and a supplement as described above (the Sx product or other variation), carried by the carrier. If using the Sx product as supplement or base for the supplement, the Sx portion includes propanoic acid and isoamyl hexanoates, at a volume ratio of 7:2. This mixture also corresponds to a molar ratio of 8.83:1, moles of propionic acid per mole of isoamyl hexanoates. The isoamyl hexanoates are formed from a ratio of esters of 99:1 of the 3 isomer to the 2 isomer. The concentration of the Sx supplement added to the animal feed may be, for example, between approximately 0.125% and approximately 0.375% by weight.

The Sx or similar supplement has the broad anti-infective properties described above, including without limitation antimicrobial, antifungal, and antiviral properties, thus promoting gut health and a boost in immunity for animals (such as when fed orally), such as in monogastric animals, ruminants and poultry, as well as for companion pet animals, through action on the animal microbiome. Additionally, the Sx or similar supplement preserves the feedstuff in storage by inhibiting pathogens that otherwise can cause mold, spoilage, and mycotoxin exposure. The animal feed composition with this supplement thus may be used to improve gut health, for weight gain, and as an immune modulator (boosting immunity to help prevent and/or treat infections). The supplement also may be used as a feed preservative. The compositions provide these benefits without being, or needing, a traditional “antibiotic.”

The composition for use as a waste treatment also could use the Sx or similar formulation. The composition may be applied to human or animal waste to decontaminate, degrade and/or deodorize the waste. As non-limiting examples, the Sx mixture may be used for the treatment of waste in latrines, cat litter boxes, animal stalls, barns, chicken-raising facilities, pig barns, pet stations in homes, and zoos. A further non-limiting example includes treating poultry litter with the Sx mixture and spreading the Sx-treated litter on top of the birds' existing litter. For example, 3 ml. of the Sx mixture could be administered per pound of litter.

These and other features of the present subject matter will become readily apparent upon further review of the following specification.

### BRIEF DESCRIPTION OF THE DRAWINGS

FIG. 1 is a graph showing the efficacy of the composition for treating infections, in a gaseous phase, in treating coronavirus disease 2019 (COVID-19).

FIG. 2A is a graph showing the efficacy of the composition for treating coronavirus disease 2019 (COVID-19) (Human USA-WA 1/2020 strain) in the gas phase between zero and six hours, post-exposure to the composition, at room temperature.

FIG. 2B is a graph showing the efficacy of the composition for coronavirus disease 2019 (COVID-19) (Human USA-WA 1/2020 strain) in the gas phase between zero and 48 hours, post-exposure to the composition, at room temperature.

FIG. 2C is a graph showing the efficacy of the composition for treating coronavirus disease 2019 (COVID-19) (Human USA-WA 1/2020 strain) in the gas phase between zero and six hours, post-exposure to the composition, at 37° C.

FIG. 2D is a graph showing the efficacy of the composition for treating coronavirus disease 2019 (COVID-19) (Human USA-WA 1/2020 strain) in the gas phase between zero and 48 hours, post-exposure to the composition, at 37° C.

FIG. 3A is a graph showing the efficacy of a composition for treating influenza (2009 pandemic H1N1 strain containing the H275Y NA mutation) in the gas phase between zero and six hours, post-exposure to the composition, at room temperature.

FIG. 3B is a graph showing the efficacy of the composition for treating influenza (2009 pandemic H1N1 strain containing the H275Y NA mutation) in the gas phase between zero and 48 hours, post-exposure to the composition, at room temperature.

FIG. 3C is a graph showing the efficacy of the composition for treating influenza (2009 pandemic H1N1 strain containing the H275Y NA mutation) in the gas phase between zero and six hours, post-exposure to the composition, at 37° C.

FIG. 3D is a graph showing the efficacy of the composition for treating influenza (2009 pandemic H1N1 strain containing the H275Y NA mutation) in the gas phase between zero and 48 hours, post-exposure to the composition, at 37° C.

FIG. 4A is a graph showing the efficacy of a composition for treating coronavirus disease 2019 (COVID-19) (Human USA-WA 1/2020 strain) in vapor form at room temperature (18° C.).

FIG. 4B is a graph showing the efficacy of a composition for treating coronavirus disease 2019 (COVID-19) (Human USA-WA 1/2020 strain) in vapor form at a temperature of 37° C.

FIG. 5 is a graph showing the effect of differing volumes of liquid in the composition for treating coronavirus disease 2019 (COVID-19) (Human USA-WA 1/2020 strain) on the ability of vapors to inactivate infectious the coronavirus causing COVID-19; i.e., SARS-CoV-2.

FIG. 6 is a graph showing the antiviral effect of differing volumes of the composition for treating coronavirus disease 2019 (COVID-19) (Human USA-WA 1/2020 strain) to inactivate infectious SARS-CoV-2.

FIG. 7A is a graph showing the antiviral effect of differing volumes of propionic acid.

FIG. 7B is a graph showing the antiviral effect of differing volumes of isoamyl hexanoates.

FIG. 8 is a graph showing the antiviral effect of differing volumes of isobutyric acid in combination with isoamyl hexanoates (esters) (at a ratio of 7:2, acid to esters) to inactivate infectious SARS-CoV-2.

FIG. 9 is a graph comparing the antiviral effect of the composition for treating coronavirus disease 2019 (COVID-19) (Human USA-WA 1/2020 strain) in vapor form against a control at room temperature.

FIG. 10 is a graph comparing the antiviral effect of the composition for treating coronavirus disease 2019 (COVID-19) (Human USA-WA 1/2020 strain) in vapor form against a control at room temperature.

FIG. 11 is a graph comparing the antiviral effect of the composition for treating coronavirus disease 2019 (COVID-19) (Human USA-WA 1/2020 strain) in vapor form against a control at differing volumes of the composition.

FIG. 12 is a graph comparing the antiviral effect of the composition for treating coronavirus disease 2019 (COVID-19) (Human USA-WA 1/2020 strain) in vapor form against a control at differing volumes of the composition.

FIG. 13 is a graph showing the antiviral effect of the composition for treating coronavirus disease 2019 (COVID-19) (Human USA-WA 1/2020 strain) at differing volumes of the composition.

FIG. 14 is a graph showing the antiviral effect of the composition for treating coronavirus disease 2019 (COVID-19) (Human USA-WA 1/2020 strain) at differing volumes of the composition.

FIG. 15 is a graph showing the antiviral effect of the composition for treating coronavirus disease 2019 (COVID-19) (Human USA-WA 1/2020 strain) at differing volumes of the composition.

FIG. 16 is a graph showing the antiviral effect of the composition for treating coronavirus disease 2019 (COVID-19) (Human USA-WA 1/2020 strain) at differing volumes of the composition and compared against a control.

FIG. 17 is a graph showing the antiviral effect of propanoic acid at differing volumes and compared against a control.

FIG. 18 is a graph showing the antiviral effect of hexamers at differing volumes and compared against a control.

FIG. 19 is a graph showing the antiviral effects of the composition for treating infections on SARS-CoV-2 as a function of time.

FIG. 20 is a graph showing the antiviral effects of propionic acid on SARS-CoV-2 as a function of time.

FIG. 21 is a graph showing the antiviral effects of isoamyl hexanoates on SARS-CoV-2 as a function of time.

FIG. 22 is a graph comparing illness scores of calves treated for Cryptosporidium with the composition for treating infections.

Similar reference characters denote corresponding features consistently throughout the attached drawings.

### DETAILED DESCRIPTION OF THE PREFERRED EMBODIMENTS

The composition for treating infections may be used for treating a wide variety of different infection and conditions, including bacterial, parasitic, protozoan, fungal and viral infections. The composition includes isoamyl hexanoates and at least one acid. The at least one acid may be lactic acid, propanoic acid, isobutyric acid, butyric acid, lactic acid, formic acid, acetic acid, citric acid, oxalic acid, uric acid, malic acid, tartaric acid or combinations thereof. A preferred embodiment is a composition, hereinafter called the “Sx” composition, which includes a 7:2 by volume mixture of propionic acid and isoamyl hexanoates. This mixture also corresponds to a molar ratio of 8.83:1 (8.83 moles of propionic acid per mole of isoamyl hexanoates). The isoamyl hexanoates is commercially available as a mix of isomers in a 99:1 ratio of the 3 isomer to the 2 isomer. Depending on the use and intended mode of delivery, the Sx composition may include or be added to other components, e.g., flavors, excipients, nutritional supplements, etc.

The composition as designed for treatment of an infection may be provided in any suitable form, including, but not limited to, a cream, an ointment, a rinse, an oil, a scrub, a spray, a shampoo, a gel, a plaster, a solution, a suspension, a dip, a salve, an ear rinse, a powder, an eyewash, mouthwash, a nail lacquer, a gas or gas phase, or an orally administered treatment. The composition may also be used as a treatment for wound care. Further, it should be understood that any suitable type of carrier may be used to administer the composition. As a non-limiting example, a molecular sieve may be used to adsorb and then desorb to administer the composition.

In use, an effective amount of the composition is administered to a patient suffering from a condition, or as a preventative of such a condition, or from diarrhea associated with the condition. The patient may be a human being or an animal. Non-limiting examples of conditions to be treated with the composition include coronavirus disease 2019 (COVID-19), influenza, and other viral infections. Additionally, the composition may be used as a treatment for biosecurity decontamination and in the processing of meat, poultry, eggs and other foodstuffs, particularly with regard to viral contaminants. In addition to the above examples, the composition may be integrated into animal feed or an animal feed supplement for an animal patient. Non-limiting examples of conditions to be treated with the composition include a fungal infection, infection with protozoa, mastitis, rain rot, scald, foot rot, an ear infection, ring worm, dermatitis, a bacterial infection, a parasitic infection, a viral infection, diarrhea, a Streptomyces avermitilis infection, vaginitis, an oral infection, a nasal infection, a throat infection, jock itch, vaginosis, a toenail infection, a fingernail infection, an eye infection, and combinations thereof.

The composition may also be used, for example, as a de-wormer or treatment for endoparasites in horses, cattle, sheep, goats, swing, dogs, cats and the like. The composition may also be used as an ingredient in a stain or odor eliminator, a urine eliminator, a cleaner or the like. Additionally, the composition may be used to treat fungal and bacterial infections, including, but not limited to, Cercospora beticola, Phytophthora cinnamomic, Verticillium dahlia, Sclerotinia sclerotiorum, Pythium ultimum, Fusarium subglutinans, Trichoderma viridae, Rhizoctonia solani, Aspergillus fumigatus, Candida albicans, Escherichia coli, Bacillus subtilis, and Saccharomyces cerevicae.

Treatment was tested on unvaccinated cattle who had succumbed to a very bad outbreak of bovine coronavirus, with infection found in approximately 400 cow/calf pairs. The outbreak of coronavirus was verified by the cattle's regular veterinarian. Infection was visibly evident, with the infected cattle appearing extremely sick with signs of coronavirus, including diarrhea and dehydration. The cattle were treated with a 1% solution of the Sx composition. Following treatment with the 1% solution of the Sx composition, the cattle recovered quickly, including rapid rehydration of the calves.

Treatment was also tested on piglets infected with the porcine epidemic disease virus (PEDv). Porcine epidemic diarrhea (PED) is caused by the PEDv coronavirus. The clinical signs of disease are very age-specific and much more severe in younger animals. In very young piglets there is profuse, watery diarrhea, without blood or mucus, which is usually yellow-green in color, often accompanied with vomiting and anorexia which may lead to death in up to 100% of the piglets less than a week old. The piglets had symptoms of dehydration and yellow diarrhea. The presence of PEDv at was confirmed by the USDA in conjunction with Newport Labs of Worthington, Minnesota. In the weeks prior to treatment with a 1% solution of the Sx composition, over 850 piglets on the ranch were lost to PEDv, which is known to have close to a 100% mortality rate.

Treatment was initially tested on one 8-day-old piglet showing yellow diarrhea. 6 ml of the 1% solution of the Sx composition was administered orally with a small syringe. The piglet showed signs of recovery after five hours and showed no evidence of diarrhea the following day. Ten 14-day-old piglets were then treated, with all showing strong evidence of diarrhea with loose, dark yellow stools. 4 ml of the 1% solution of the Sx composition was administered to each piglet. Each animal had recovered within 24 hours.

Another ten 3-day-old piglets were treated after showing signs of PEDv. Each piglet was treated with 2 ml of the 1% solution of the Sx composition and diarrhea completely ceased. After several weeks, all of the piglets remained alive. Another thirty 3-day-old piglets, showing signs of diarrhea, were also treated with 3 ml of the 1% solution of the Sx composition. All of the piglets remained alive following treatment. Samples were taken from the piglets and confirmed by Norwalk Labs as being free from PEDv. There was also no reoccurrence of diarrhea or other symptoms of PEDv in the piglets.

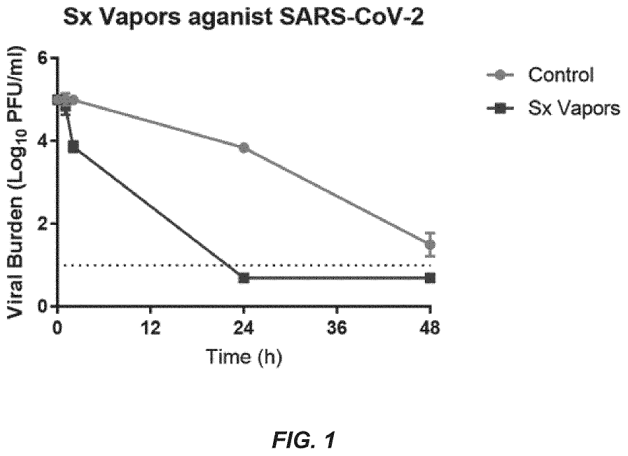

FIG. 1 is a graph showing the efficacy of the Sx composition, in a gaseous phase, in treating coronavirus disease 2019 (COVID-19). As can be seen, the Sx composition, in vapor or gaseous form, is effective at dropping viral load within 2 hours, and after 24 hours, the viral load is severely depleted, dropping close to zero. In the experiment with the Sx composition, the solution vaporized and acted on the virus in the gaseous phase, with all safety precautions taken. The temperature was 37° F. throughout the course of the testing. A novel gas assay test was conducted in which 100 ml of the Sx composition was placed in a plastic centrifuge detached lid in the middle of a Petri plate having a volume of 100 ml. Small plates with SARS-cov-2 virus were placed in the same Petri plate and then sealed with parafilm. At least five plates were prepared in this manner. At various time intervals, the plates with the virus were assayed in the mammalian cell line standard test for the production of platelets, with each one theoretically representing one infectious active virus particle. As can be seen in the graph, which is log-scale on the horizontal axis, there is an immediate rapid reduction in active virus particles. Within 24 hours, there is nearly a 5-log reduction in active virus particles. The Sx product may be administered in its gaseous or vapor phase using any suitable type of vaporizing device, such as a nebulizer or the like, for example. Toxicity of a 1% solution of the Sx composition has been tested in dogs, with no toxicity having been found.

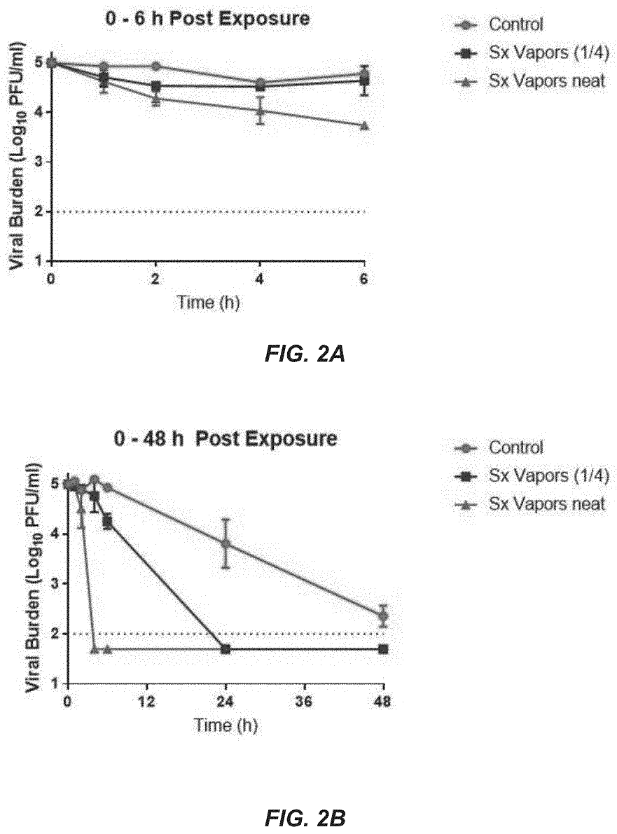

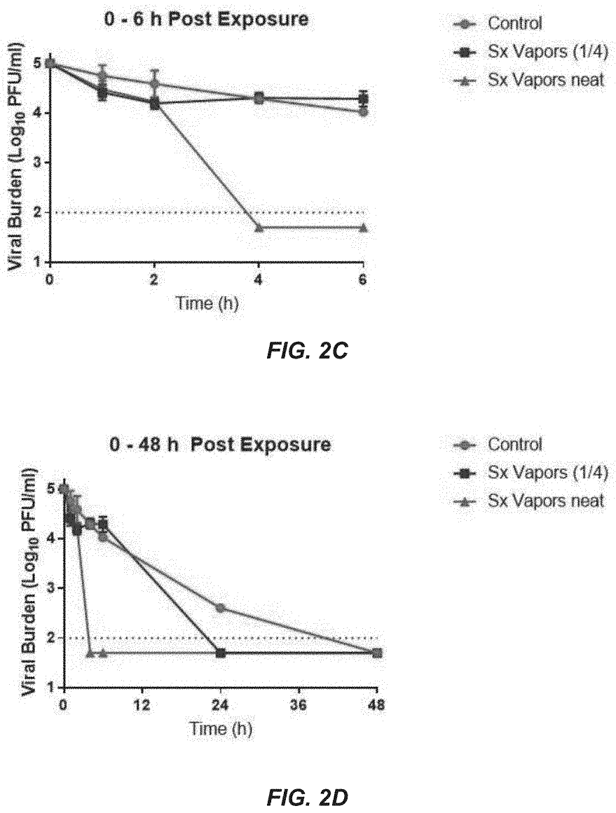

FIGS. 2A and 2B are graphs showing the efficacy of the Sx composition, in the gaseous phase, in treating coronavirus disease 2019 (COVID-19) (Human USA-WA 1/2020 strain), shown for 0-6 hours post-application and 0-48 hours post-application, respectively. The Sx composition was tested as a concentrated mixture (i.e., “neat”) and as a diluted mixture of 1 part Sx neat to 4 parts tissue culture medium. In FIGS. 2A and 2B, the results are shown for effectiveness at room temperature. FIGS. 2C and 2D are also graphs showing the efficacy of the Sx composition, in the gaseous phase, in treating coronavirus disease 2019 (COVID-19) (Human USA-WA 1/2020 strain), shown for 0-6 hours post-application and 0-48 hours post-application, respectively. In FIGS. 2C and 2D, the results are shown for effectiveness at 37° C. (i.e., average healthy human body temperature). For each experiment, 50 μL of the Sx composition (neat and diluted) was tested against 1 mL containing 10⁵ plaque forming units (PFU) of virus in a petri dish. One petri dish was used per time point and the samples were assayed in duplicate. The infectious viral burden was determined by plaque assay on Vero E6 cells.

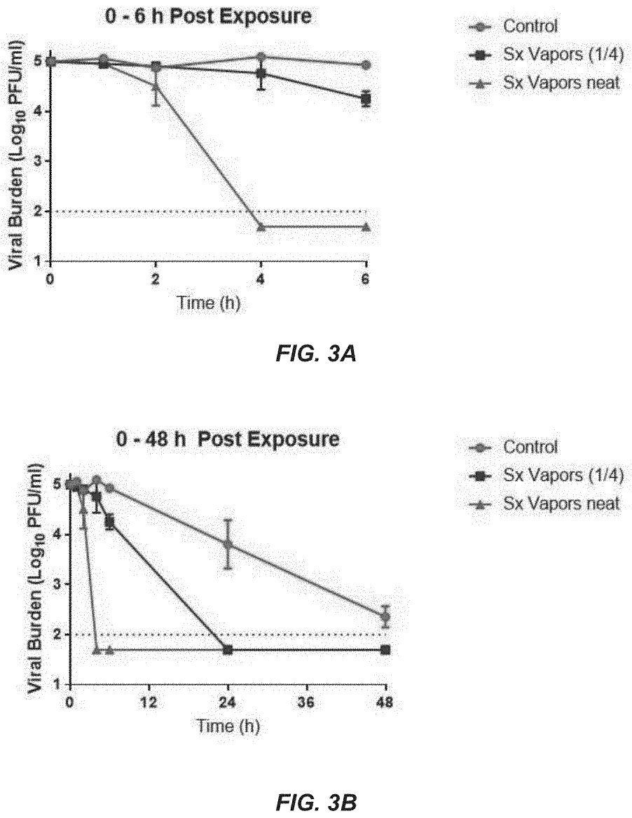

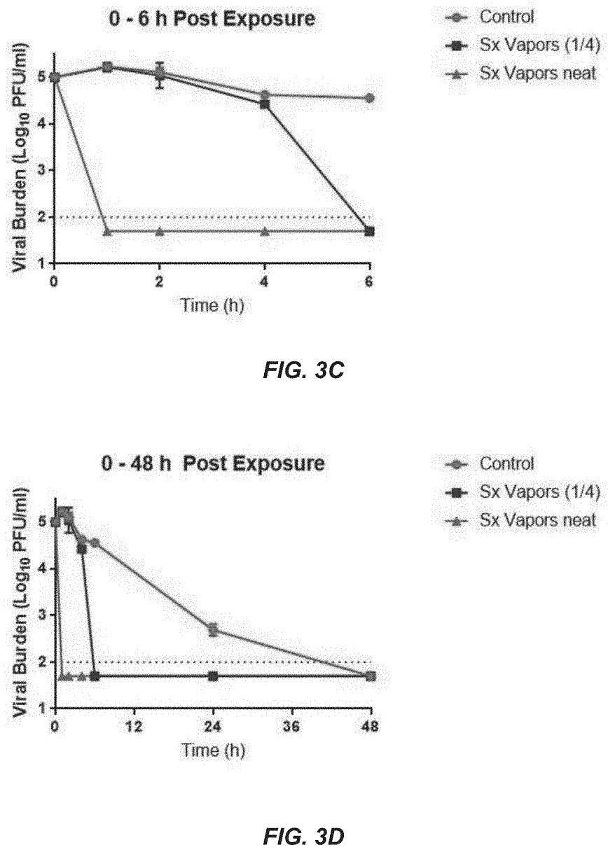

Additionally, FIGS. 3A and 3B are graphs showing the efficacy of the Sx composition, in the gaseous phase, in treating influenza (2009 pandemic H1N1 strain containing the H275Y NA mutation, which confers resistance to oseltamivir), shown for 0-6 hours post-application and 0-48 hours post-application, respectively. The Sx composition was tested as a concentrated mixture (i.e., “neat”) and as a diluted mixture of 1 part Sx neat to 4 parts tissue culture medium. In FIGS. 3A and 3B, the results are shown for effectiveness at room temperature. FIGS. 3C and 3D are also graphs showing the efficacy of the Sx composition, in the gaseous phase, in treating influenza (2009 pandemic H1N1 strain containing the H275Y NA mutation, which confers resistance to oseltamivir), shown for 0-6 hours post-application and 0-48 hours post-application, respectively. In FIGS. 3C and 3D, the results are shown for effectiveness at 37° C. For each experiment, 50 μL of the Sx composition (neat and diluted) was tested against 1 mL containing 10⁵ plaque forming units (PFU) of virus in a petri dish. One petri dish was used per time point and the samples were assayed in duplicate. The infectious viral burden was determined by plaque assay on MDCK cells.

Returning to the treatment of COVID-19, the Sx composition was prepared as a mixture of propionic acid and isoamyl hexanoates at a ratio of 7:2 (v/v). The isoamyl hexanoates were present in the mixture of esters at a ratio of 99:1 of the 3 isomer to the 2 isomer. This particular mixture is hereinafter referred to as the “Propylamylatin” ™ formula. For purposes of testing, Vero E6 cells were purchased from the American Type Culture Collection (ATCC; Manassas, VA, USA) and cultured in Minimum Essential Medium (MEM; Corning Cellgro, Tewksbury, MA, USA) supplemented with 10% fetal bovine serum (FBS; Hyclone, Logan City, UT, USA) and 1% penicillin-streptomycin solution (Hyclone). Cells were maintained at 37° C., 5% CO2 and sub-cultured twice weekly to ensure sub-confluency. The USA-WA1/2020 strain of SARS-CoV-2 was deposited by the Centers for Disease Control and Prevention and obtained through BEI Resources, NIAID, NIH (NR-52281). Viral stocks were propagated in Vero E6 cells for two days. Supernatants were collected, clarified by high-speed centrifugation, aliquoted and frozen at −80° C. Viral stock titers were determined by plaque assay on Vero E6 cells. All work with SARS-CoV-2 was conducted under Biosafety Level 3 plus conditions at the Institute for Therapeutic Innovation (University of Florida, Orlando, FL) using safety protocols approved by the University of Florida Institutional Biosafety Committee.

For vapor (gas phase) evaluations of the Propylamylatin™ formula, SARS-CoV-2 was diluted to a concentration equivalent to 10⁵ plaque forming units (PFU)/ml in MEM. Aliquots containing 1 ml of the virus suspension (10⁵ PFU) were placed into plastic caps and secured to the surface of 100 mm×15 mm polystyrene petri dishes with double sided tape directly in the middle of the petri dishes. Small plastic caps (the lid from a standard microcentrifuge tube) containing 100 μls of the Propylamylatin™ formula were placed 2.5 cm away from the virus-containing caps. The top of the petri dish was sealed with parafilm and plates were incubated at both room temperature (˜18° C.) and 37° C. for various lengths of time. Since the removal of the top cover of the petri dish immediately destroys the gas atmosphere of that dish, one petri dish for each experimental arm was harvested at each time point. Time-matched control plates were included in the experimental design and set up exactly as described above with the exception that 100 μls of PBS was placed inside the white small plastic cap. At the time of sampling, viral supernatant was transferred from the plastic cap into a microfuge tube and frozen at −80° C. until the end of the study. Viral burden was quantified by plaque assay on Vero E6 cells for all samples simultaneously. Each experimental arm was assayed in duplicate to determine between sample variability and at least two independent assays were conducted on different days to address between day variability.

Additional vapor (gas phase) evaluations were conducted with various volumes of the Propylamylatin™ formula against SARS-CoV-2 at 37° C. Studies were conducted as described above, but for these experiments different volumes ranging from 25 μls to 100 μls of the Propylamylatin™ formula were placed in the white small plastic cap. Time and volume matched PBS controls were included in this experiment. All experimental arms were sampled in duplicate and processed as described above. Viral burden was determined by plaque assay on Vero E6 cells and two independent studies were conducted on two different days.

The ability of the Propylamylatin™ formula to inactivate SARS-CoV-2 when directly injected into a viral suspension was also evaluated. For these studies, various volumes of the Propylamylatin™ formula were directly inoculated into 1 ml aliquots of SARS-CoV-2 diluted to a concentration of 10⁵ PFU/ml in MEM and incubated at 37° C. At various times post-inoculation, aliquots from each experimental arm were harvested, in duplicate, and frozen at −80° C. until the end of the study. Viral burden was quantified by plaque assay on Vero E6 cells for all samples simultaneously. At least two independent studies were conducted on different days to address between-day variability.

The anti-SARS-CoV-2 activity of the individual components making up the Propylamylatin™ formula (i.e.: propionic acid alone and esters alone) were evaluated in the amounts in which they are present in the formula at the 7:2 (v/v) ratio, using the direct injection methods described above. Isobutyric acid (the original acid produced by M. albus) was substituted for the propionic acid in the mixture and also assessed via direct injection.

Vero E6 cells were seeded into 6-well plates and incubated until confluency at 37° C., 5% CO2. Viral samples were thawed on ice, serially diluted 10-fold in MEM, and 100 μls of each dilution was inoculated onto Vero E6 monolayers. Plates were incubated for 1 h to allow for viral attachment and were shaken every 15 mins to maintain an even distribution of the viral inoculum on the cell monolayer. Monolayers were then overlaid with MEM containing a final concentration of 0.5% agar (w/v) and 5% FBS (v/v). Two days later, a secondary overlay containing MEM with a final concentration of 0.5% agar, 1% FBS, and 0.007% neutral red solution was added to each well of the 6-well plate. Plaques were counted on the following day (72 h post-infection) with the naked eye.

The amount of dissolved propionic acid and isoamyl hexanoates in MEM was estimated when the Propylamylatin™ formula was administered via gas phase, and the resulting concentrations served as a guide for the design of the direct injection experiments. For these assays, petri dishes were set up exactly as described above for the Propylamylatin™ formula gas phase evaluations with the exception that virus was not included these studies. Propionic acid was quantified using two different methods. The first method was via titration of the MEM (which contains phenol red, a pH indicator) with a 1 M NaOH solution. The second method was by GC/FID in which samples, taken from the wells, were diluted 20-fold, separated by GC, and the amounts measured by FID. The samples were injected into a Hewlett Packard 6890 gas chromatograph containing a 30 m×0.25 mm inner diameter ZB Wax capillary column with a film thickness of 0.50 mm. A thermal program of 30° C. for 2 min followed by an increase to 220° C. at 5° C. min-1 was applied. Ultrahigh purity helium gas was used as the carrier gas and the initial column head pressure was 50 KPa. Quantification for both methods were conducted in triplicate.

The presence of the esters was determined both qualitatively and quantitatively by GC/MS using a Hewlett Packard 6890 gas chromatograph. The samples were diluted 2-fold and the initial identification of the esters found in the analysis was made via library comparison using the National Institute of Standards and Technology (NIST) database. For GC/MS analyses, authentic samples of isoamyl hexanoates were used to generate standard curves to interpolate the concentrations of each compound in the media samples.

Various volumes of the Propylamylatin™ formula were added to 1 ml of MEM. The pH of the media was measured using MColorpHast pH test strips and indicator papers (MilliporeSigma, Burlington, MA) at various times ranging from 15 min to 24 h post- the Propylamylatin formula addition.

The volume of the Propylamylatin™ formula that reduces viral burden by 50% (EC₅₀) was determined on individual time points as well as over the entire duration of the experiment. For analyses at individual time points, an inhibitory Sigmoid-Emax model (Hill model) was fit to the viral burden data from each experimental arm generated at that time point. Analyses of the entire data set was determined by first calculating the area under the viral burden time curve (AUC_{VB-time}) for each experimental condition. An inhibitory Sigmoid-Emax model (Hill model) was then fit to the AUC_{VB-time} values. All inhibitory Sigmoid-Emax models were fit to the data using GraphPad Prism software (version 7.02).

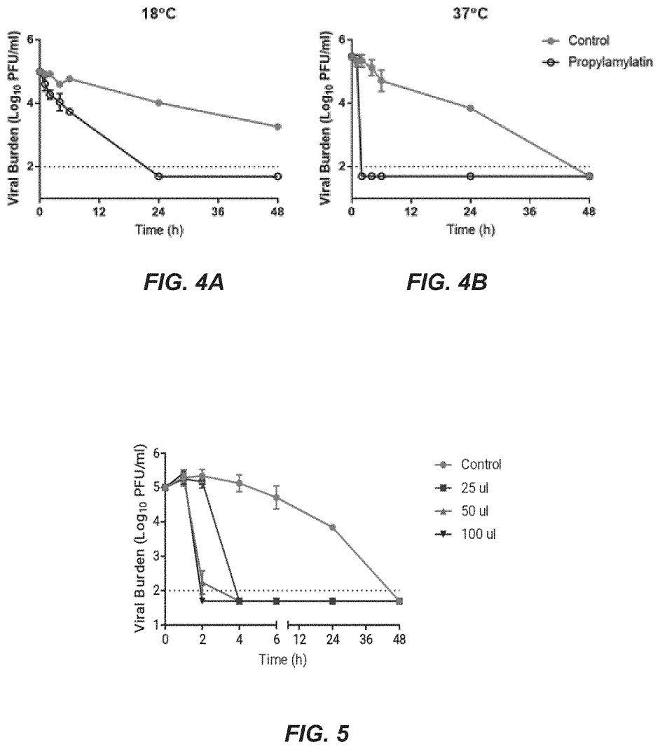

The ability of Propylamylatin™ formula vapors to inactivate infectious SARS-CoV-2 at room temperature (˜18° C.) and body temperature (37° C.) was first examined. SARS-CoV-2 naturally loses infectivity over time, as viral burden decreased in the control arms at both temperature conditions (see FIGS. 4A and 4B). Viral stability, however, was heavily influenced by temperature. At 18° C., viral burden decreased by 1-log₁₀ PFU/ml at 24 h and by 1.7-log₁₀ PFU/ml at 48 h in the control arm (FIG. 4A). In contrast, control titers were reduced by 1.7-log₁₀ PFU/ml at 24 h and were undetectable at 37° C. after 48 h (FIG. 4B). These findings indicate that cooler temperatures allow for a longer period of infectivity for SARS-CoV-2.

Propylamylatin™ formula vapors were effective at neutralizing infectious SARS-CoV-2 and exposure to these vapors resulted in viral burden levels below the assay limit of detection for both temperature conditions at time points that were markedly faster than those observed for the corresponding controls (FIGS. 4A and 4B). The duration of the Propylamylatin™ formula exposure to achieve complete viral inactivation (where titers were below the limit of detection) was heavily influenced by temperature. Inactivation of SARS-CoV-2 was markedly more rapid at 37° C., as infectious virus was undetectable after only 2 h of exposure to the Propylamylatin™ formula vapors (FIG. 4B). The reduction in viral burden was more gradual at 18° C. and 24 h of exposure time was required to completely inactivate infectious virus at room temperature (FIG. 4A). These findings demonstrate that the Propylamylatin™ formula vapors are effective at neutralizing infectious SARS-CoV-2 and neutralization is faster at higher temperatures.

Experiments were also conducted to evaluate the influence of the Propylamylatin™ formula volume on the ability and timing of the vapors to inactivate SARS-CoV-2 at 37° C. Vapors stemming from all starting liquid volumes of the Propylamylatin™ formula demonstrated potent anti-SARS-CoV-2 activity, driving viral burden to levels below the assay limit of detection (FIG. 5). However, the duration of time required to achieve complete inactivation of infectious SARS-CoV-2 was directly related to the liquid volume of the Propylamylatin™ formula at the start of the experiment (FIG. 5). Larger volumes of Propylamylatin™ formula resulted in lower viral loads faster than smaller volumes. This was most evident at the 2 h post-exposure time point, as experimental arms with 25 μl of the Propylamylatin™ formula exhibited viral titers that were similar to the control (5.2-log₁₀ PFU/ml) whereas arms with 50 and 100 μls of liquid had viral titers that were near (2.2-log₁₀ PFU/ml) or below the plaque assay limit of detection, respectively (FIG. 3). The volume of the Propylamylatin™ formula that yields an effective concentration of vapors to reduce SARS-CoV-2 burden by 50% (EC₅₀) was 38.81 μls at the 2 h time point. Infectious SARS-CoV-2 was not detected in any of the Propylamylatin™ formula experimental arms by 4 h post-exposure.

The amounts of dissolved propionic acid and isoamyl hexanoates in tissue culture media (absent of virus) that were exposed to vapors volatilizing from various volumes of Propylamylatin™ formula over time when incubated at 37° C. were measured. These experiments were performed to identify an exposure-response relationship between the propionic acid and/or isoamyl hexanoates and antiviral effect. The evaluations were focused on the early time points, as viral burden was below the limit of detection in all of the Propylamylatin™ formula vapor-treated arms after 4 h of exposure. As expected, higher levels of propionic acid were detected when larger volumes of the Propylamylatin™ formula were allowed to volatilize and levels increased over time (Table 1 below). The Propylamylatin™ formula volume-dependent response was observed only at 2 h post-vapor exposure due to the fact that virus was undetectable at all subsequent time points with the exception of the control (FIG. 5). At 2 h, 2.6 μls of dissolved propionic acid was detected in the 25 μl experimental arm whereas 6.1-7.7 μls and 9.7-22.5 μls (depending on the quantification method used) was detected in the 50 and 100 μl experimental arms, respectively. By 4 h post-vapor exposure (when all experimental arms had undetectable viral titers), propionic acid levels were between 5.5-6.4 μls when 25 μls of the Propylamylatin™ formula was volatilized, 16.2 μl when 50 μl volatilized, and 20.5 μls when 100 μls was volatilized. Isoamyl hexanoates were not detected in medium that was incubated in the presence of 25 or 50 μls of evaporated the Propylamylatin™ formula and existed at low levels (1.4 μls) when 100 μls was allowed to evaporate (Table 1 below).

TABLE 1
The amount of dissolved propionic acid and isoamyl hexanoates in
tissue culture medium incubated in the presence of volatilized
Propylamylatin ™ formula resulting from different liquid volumes

| | Time post- | Amount of | | Amount of |
|---|---|---|---|---|
| Amount of | exposure to | Propionic acid | Amount of | Isoamyl |
| Propylamylatin ™ | Propylamylatin | via titration | Propionic acid | Hexanoates |
| formula | formula vapors | with 1M NaOH | via GC/FID^{a} | via GC/MS^{b} |
| (μl) | (h) | (μls) | (μls) | (μls) |
| 25 | 2 | 2.6 | ND^{c} | ND^{c} |
| | 4^{d} | 5.5 | 6.4 | 0 |
| | 6 | 8.6 | ND^{c} | ND^{c} |
| 50 | 2 | 7.7 | 6.1 | 0 |
| | 4^{d} | 16.2 | ND^{c} | 0 |
| | 6 | 22 | ND^{c} | 0 |
| 100 | 2^{d} | 9.7 | 22.5^{e} | 1.4 |
| | 4 | 20.5 | ND^{c} | ND^{c} |
| | 6 | 25.5 | ND^{c} | ND^{c} |

^{a}Gas chromatography/Flame Ionization Detection (GC/FID)
^{b}Gas chromatography/Mass Spectroscopy (GC/MS)
^{c}ND: Not Done
^{d}The viral burden was below the assay limit of detection

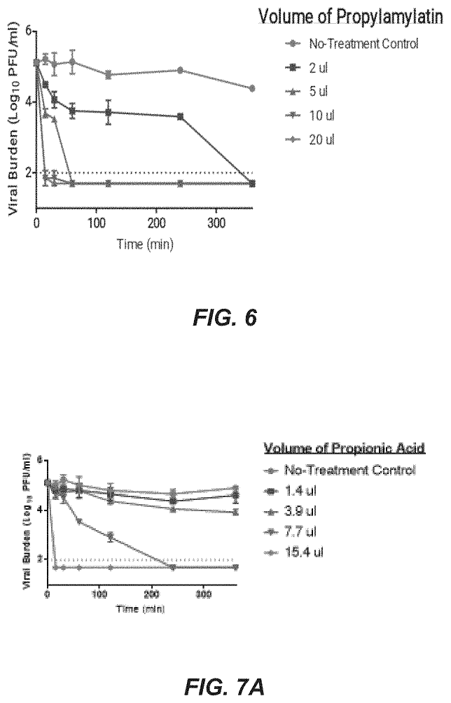

The Propylamylatin™ formula completely inactivated infectious SARS-CoV-2 virus when directly added to viral suspensions; however, the rapidity of inactivation was heavily influenced by the amount of formula administered (FIG. 6). The addition of 10 and 20 μls of the Propylamylatin formula resulted in nearly undetectable levels of infectious virus after just 15 mins of exposure, whereas viral burden was equivalent to 3.7- and 4.5-log₁₀ PFU/ml after the addition of 5 μl and 2 μls, respectively, at the same time point. Viral burden was below the assay limit of detection at 60 mins following the addition of 5 μls of the Propylamylatin™ formula and at 360 mins (6 h) after adding 2 μls of agent. SARS-CoV-2 infectivity was stable throughout the experiment with viral titers ranging from −5-log₁₀ PFU/ml to 4.5-log₁₀ PFU/ml over the 6 h study, suggesting that any loss of viral infectivity is due to the Propylamylatin™ formula and not natural degradation.

The EC₅₀ volume of the Propylamylatin™ formula was calculated at individual time points as well as over the duration of the entire experiment using Hill models. The EC₅₀ estimates decreased as exposure time increased, resulting in a value of 6.47 μls at 15 mins, 5.80 μls at 30 mins, and ˜2.02 μls at 60 mins to 240 mins. The EC₅₀ value for the entire 360 min study was determined to be 2.10 μls. These findings demonstrate the influence of exposure time on the antiviral activity of the Propylamylatin™ formula as well as the potency of this agent to inactivate infectious SARS-CoV-2.

The addition of the Propylamylatin™ formula to the viral diluent medium resulted in a clear color change of red to yellow, signifying a change in pH, and this change was dependent on the volume of agent added. The influence of the Propylamylatin™ formula over time on the pH of the medium used to dilute the virus in the direct injection studies was characterized. The pH decreased with increasing volumes of Propylamylatin™ formula, with a final value of 8.5 in the control, 6.0 in the 2 μl arm, 5.0 in the 5 μl arm, and 4.0 in the 10 μl and 20 μl arms (Table 2 below). The pH values were stable in all arms over the 24 h experiment, with the exception of the 10 μl experimental arm which started at a pH equivalent to 4.5 at 15 min post-exposure before finally reaching a stabilized (maximal) value of 4.0 after 30 mins (Table 2). These findings indicate that the Propylamylatin™ formula creates an acidic environment in the medium upon addition.

TABLE 2
The pH of tissue culture medium used to dilute
SARS-CoV-2 at various times post-exposure
to various volumes of Propylamylatin formula

| Amount of | Time post-exposure to the | | | | | | |
|---|---|---|---|---|---|---|---|
| formula | Propylamylatin ™ formula (mins) | | | | | | |
| Propylamylatin ™ | | | | | | | 1,440 |
| (μl) | 15 | 30 | 60 | 120 | 240 | 360 | (24 h) |
| 0 | 8.5 | 8.5 | 8.5 | 8.5 | 8.5 | 8.5 | 8.5 |
| 2 | 6.0 | 6.0 | 6.0 | 6.0 | 6.0 | 6.0 | 6.0 |
| 5 | 5.0 | 5.0 | 5.0 | 5.0 | 5.0 | 5.0 | 5.0 |
| 10 | 4.5 | 4.0 | 4.0 | 4.0 | 4.0 | 4.0 | 4.0 |
| 20 | 4.0 | 4.0 | 4.0 | 4.0 | 4.0 | 4.0 | 4.0 |

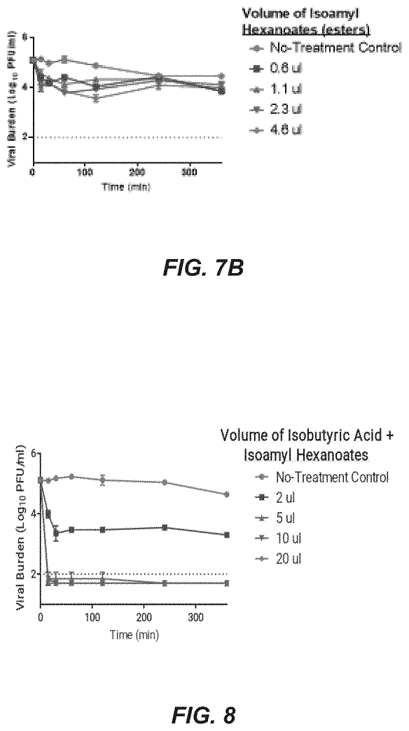

The antiviral activity of each of the components that make up the Propylamylatin™ formula was evaluated against SARS-CoV-2 individually to determine the contribution of antiviral effect from each component. Propionic acid and isoamyl hexanoates (esters) were examined as monotherapy at the proportions in which they exist in the formula, which is equivalent to a 7:2 (v/v) ratio of propionic acid to esters. For example, a 20 μl volume of the Propylamylatin™ formula consists of 15.4 μls of propionic acid and 4.6 μls of esters whereas a 2 μl volume of the Propylamylatin™ formula is comprised of 1.4 μls and 0.6 μls of propionic acid and esters, respectively. Propionic acid alone inhibited SARS-CoV-2 in an exposure-dependent manner, with larger volumes of acid providing a greater and more rapid decline in infectious viral burden (FIG. 7A). The largest volume evaluated, 15.4 μls of propionic acid, resulted in an undetectable viral burden 15 mins after exposure. The 7.7 μl volume provided a steady decline in infectious SARS-CoV-2, yielding undetectable levels of virus after 240 min (4 h) of exposure (FIG. 7A). The smallest volumes provided markedly less inhibition resulting in a decline of 0.98-log₁₀ PFU/ml with 3.9 μls and 0.29-log₁₀ PFU/ml with 1.4 μls after 360 mins post-addition. The EC₅₀ value over the entire study is 6.2 μls for propionic acid.

The isoamyl hexanoates alone were substantially less effective at suppressing SARS-CoV-2 replication and the degree of inhibition was similar between all treatment arms, indicating the lack of an exposure-response relationship (FIG. 7B). Thus, an EC₅₀ value was not calculated for this data set. The majority of inhibition occurred during the first 1 h of exposure to the esters, as viral titers were relatively unchanged at the subsequent time points. These findings suggest that the propionic acid is the more active component in the Propylamylatin™ formula. But the addition of the esters to the propionic acid is necessary, as the combination of these two components (as the Propylamylatin™ formula) provides substantially greater viral inhibition than either component individually (FIGS. 6, 7A and 7B). This is particularly evident at the lower the Propylamylatin™ formula volumes (2 μls and 5 μls) which completely suppressed infectious SARS-CoV-2 (FIG. 6), but the individual components of 1.4 μls and 3.9 μls of propionic acid and 0.6 μls and 1.1 μls of isoamyl hexanoates resulted in minimal reduction of viral burden (FIGS. 7A and 7B).

The anti-SARS-CoV-2 activity of isobutyric acid was evaluated, the original product produced by the Muscodor sp., in combination with the isoamyl hexanoates that comprise the Propylamylatin formula to determine how the substitution of isobutyric acid with propionic acid influences antiviral effect of the Propylamylatin™ formula via direct injection assays. The same ratio of 7:2 acid to esters was utilized for these studies. Similar to the Propylamylatin™ formula, isobutyric acid in combination with the isoamyl hexanoates rapidly inactivated infectious SARS-CoV-2 (FIG. 8). The isobutyric acid+ester mixture at volumes of 5, 10, and 20 μls yielded viral titers at or below the assay limit of detection after only 15 mins of exposure. In the 2 μl arm, viral titers were reduced 10-fold yielding a viral burden of 4.0 log₁₀-PFU/ml at the same time point. Infectious virus further declined to ˜3.5-log₁₀ PFU/ml by 30 mins post-administration and titers remained at this level for the duration of the experiment (FIG. 8). These findings demonstrate that the inactivation of SARS-CoV-2 by isobutyric acid in combination with the esters is heavily influenced by volume and exposure time, as also described for the Propylamylatin™ formula (FIG. 6). The degree of viral inhibition is very similar between the isobutyric acid/ester combination and the propionic acid/ester combination (Propylamylatin™ formula). The overall EC₅₀ volume for the isobutyric acid+ester mixture was 1.45 μls, which is comparable to the 2.10 μls reported for that of the Propylamylatin™ formula.

The effect of the isobutyric acid and esters in combination on the pH of the viral diluent media was also measured. pH levels for this combination were identical to those reported for the Propylamylatin™ formula over time (Table 2; data not shown). These findings indicate that the isobutyric acid and esters in combination create an acidic environment that is identical to the Propylamylatin formula upon addition to the medium used to dilute SARS-CoV-2.

The COVID-19 pandemic continues to threaten global public health, spreading readily from person-to-person and resulting in significant illness and an unacceptably high number of deaths. Although numerous preclinical and clinical investigations have been conducted to identify effective drug candidates for the treatment and prevention of COVID-19, there are currently no antiviral agents approved for this serious disease. Natural products derived from plant, fungi, and marine sources have been, and are still, frequently used to treat viral infections, particularly those that cause respiratory illnesses such as rhinovirus and influenza virus. Natural products are attractive therapies for viral, as well as other microbial infections, because they are often accessible in nature (making them low in cost), amenable to mass production, and have generally good safety profiles. Moreover, the lack of available antiviral drugs for many ubiquitous viral diseases makes natural-based remedies a logical source of treatment for these infections, especially in developing countries. For these studies, we aimed to evaluate the Propylamylatin™ formula against SARS-CoV-2, in an attempt to identify a potentially effective treatment strategy to combat COVID-19.

Because the antimicrobial activity of the natural fungus-derived VOCs was originally observed in the gas phase, we first evaluated the Propylamylatin™ formula in the gaseous state against SARS-CoV-2 at room temperature (18° C.) and body temperature (37° C.). This initial study was conducted in order to determine if the Propylamylatin™ formula might possess any antiviral activity and if so, at what concentrations. Ultimately, this information would serve as a guide for more comprehensive and better controlled studies using a direct injection method of the Propylamylatin™ formula into viral suspension. Infectious SARS-CoV-2 virus is naturally more stable at cooler temperatures with viral burden persisting longer at room temperature compared to body temperature. The Propylamylatin™ formula vapors were effective against SARS-CoV-2 at both temperatures, resulting in undetectable levels of virus after exposure. However, viral inactivation was substantially faster at higher temperatures. This is likely due to the fact that the Propylamylatin™ formula volatizes more rapidly at higher temperatures. The impressive ability of the formula vapors to inactivate high levels (˜10⁵ PFU/ml) of SARS-CoV-2 at room temperature has important implications, as this agent may serve as a way to inactivate virus on different surfaces including instruments and personal protective equipment. Studies evaluating the disinfecting potential of the Propylamylatin™ formula vapors against SARS-CoV-2 on surfaces made of plastic, glass, metal, etc. are currently ongoing.

Measuring the levels of dissolved propionic acid and isoamyl hexanoates in the tissue culture medium post-exposure to the vapors volatizing from the Propylamylatin™ formula at 37° C. revealed that propionic acid was readily detected but the esters (isoamyl hexanoates) were only present (at very low levels) when high starting volumes of compound were employed. These findings indicate that the majority of anti-SARS-CoV-2 activity was likely due to the propionic acid under this route of administration.

Per our hypothesis, our initial interest in the Propylamylatin™ formula was as a potential antiviral strategy against the virus causing COVID-19; thus, we conducted direct injection experiments where the Propylamylatin™ formula was directly added to a SARS-CoV-2 suspension at 37° C. to simulate body temperature. The addition of at least 10 μls of the Propylamylatin formula into 1 ml suspension of 10⁵ PFU of SARS-CoV-2 completely inactivated SARS-CoV-2 to levels below the limit of detection after only 15 mins of exposure. Assessing the individual components of the Propylamylatin formula revealed that indeed, as identified from the gas phase experiments, the propionic acid provided the majority of antiviral effect compared to the isoamyl hexanoates. However, the degree of inhibition was markedly greater when the propionic acid and the isoamyl hexanoates were combined, indicating synergy. These findings for antiviral activity are in concordance with what has been reported for the antimicrobial activity of the Propylamylatin formula against bacteria and fungi.

Isobutyric acid is the small weight molecular acid identified in the original VOCs made by the fungus. Isobutyric acid in combination with the isoamyl hexanoates was slightly overall more potent than the Propylamylatin™ formula (which contains propionic acid with the isoamyl hexanoates) yielding EC₅₀ values of 1.45 μls and 2.10 μls, respectively. This difference in EC₅₀ is mainly attributed to the 5 μl volume of the isobutyric mixture which completely inhibited SARS-CoV-2 after 15 mins of exposure, yielding inhibitory kinetics that were identical to the 10 μl and 20 μls volumes. A 5 μl volume of the Propylamylatin formula provided complete inhibition after 60 min of exposure. These differences in antiviral activity are relatively negligible. Moreover, there are several benefits to using propionic acid over isobutyric acid, the most obvious being the unattractive odor of isobutyric acid which smells like rancid butter. Propionic acid also has an unpleasant odor, but it is less pungent than that of isobutyric acid.

The degree and rapidity of viral inactivation via the Propylamylatin™ formula is impressive. It is hypothesized that the agent may inactivate the virus via interaction with the receptor binding domain (RBD) of the SARS-CoV-2 spike protein, which plays a critical role in viral attachment to the cellular angiotensin-converting enzyme 2 (ACE2) receptor. A polybasic cleavage site that is 10 nm from the RBD has been identified on the spike protein and shown to enhance the binding affinity of RBD to ACE2. Blocking this positively charged site (arginine) with negatively charged anions in the presence of a strong lipophilic agent should effectively neutralize this critical site and render it ineffective. This phenomenon has been previously shown, as the addition of a synthetic peptide GluGluLeuGlu to SARS-CoV-2 reduced the binding affinity of RBD to ACE2 by 34%. Reduction of binding strength would feasibly reduce the infectivity of the virus. Thus, this may be one mechanism by which the Propylamylatin formula inactivates SARS-CoV-2. A second mechanism by which this agent may is act is through its ability to create an acidic environment. The pH was inversely proportional to antiviral activity, with lower pH values resulting in a more rapid decline in viral burden. Others have reported that the infectivity of coronaviruses including SARS-CoV (causative agent of the 2002-2003 SARS outbreak) are very sensitive to pH extremes, as lower pH values (i.e., increased acidity) were able to completely inactivate SARS-CoV. Thus, the Propylamylatin™ formula may rapidly inactivate infectious SARS-CoV-2 via blocking the polybasic cleavage site on the spike protein and/or creating an acidic environment that renders the virus non-infectious.

Our studies illustrate the therapeutic potential of the Propylamylatin™ formula for the treatment of COVID-19. As shown above, a volume of only 10 μls was required to completely inactivate infectious SARS-CoV-2 after only 15 min of contact time. This amount of formula is equivalent to 1% of the total volume of liquid in the suspension (10 μls of the Propylamylatin™ formula into 1,000 μls of virus suspension) and represents the exact amount of the Propylamylatin formula that was present in the electrolyte solution originally administered to piglets suffering with PEDv. These findings warrant further investigations to determine if the level of the Propylamylatin™ formula required to inactivate SARS-CoV-2 is physiologically achievable in humans and whether it will have direct application to treat human COVID-19.

Gastrointestinal symptoms have been reported in patients with COVID-19, and SARS-CoV-2 has been shown to infect human gut enterocytes. Therefore, a 1% solution of Propylamylatin formula may be sufficient to inhibit viral replication in the digestive system thereby alleviating gastrointestinal symptoms. Respiratory symptoms may also be relieved with the formula. The Propylamylatin™ formula could possibly be applied as a topical agent in the nasal cavities to inactivate infectious virus in the nose, which is the initial site of infection. It has been proposed that aspiration from the nasal cavity into the oral cavity may be a route for the virus to transverse from the upper respiratory system into the lower respiratory system, resulting in more severe disease outcomes. Thus, a 1% solution of the Propylamylatin™ formula as a mouthwash may help inhibit any virus in the oral cavity from reaching the lungs, thereby preventing pneumonia as well as other more severe disease outcomes. Finally, Propylamylatin™ formula when administered via an atomizer, nebulizer, or intubation directly into lung tissue either orally or nasally may be helpful in reducing viral load in these tissues. Additional studies are required to address these hypotheses.

In addition to the above, the Sx composition has also been used to treat and deactivate SARS-CoV-2 using direct injection of an antiviral mixture containing the Sx composition. Viral assays were prepared by suspending SARS-CoV-2 virions in MEM 1 X Eagles medium and plaque assays to detect the active virus in the wild Vero E6 human cell line. Treatments of the virus suspensions were initially performed using the gas phase of Sx as a mimic to learn which concentrations of Sx may be biologically effective. To this end, 1 mL of a suspension of the virus containing ca 100,000 active virions was placed in a plastic cap and held tightly to the surface of a Petri plate with modeling clay. Another small plastic cap holding the Sx mixture was placed 2.5 cm away from the first cap holding the viral suspension, in the center of the plate. The top of the Petri plate was then sealed with a parafilm and the plate was incubated at both 20° C. and 37° C. for varying lengths of time. Each time point measured in the gas assay represented one complete plate, since removal of the top cover of the plate immediately destroyed the gas atmosphere of that plate.

Once an idea of effective concentrations was obtained, varying amounts of the Sx mixture was directly injected into the 1 mL buffered virus suspension at different amounts and under different time frames of exposure at 37° C., which best mimics the human temperature. Additionally, the effects of the acid alone and the esters alone of the Sx mixture were measured in the amounts in which they are present (i.e., at the 7:2 v/v ratio). Then, other acids, including isobutyric acid and acetic acid, were substituted for the propionic acid in the mixture and tested in order to learn if other acids, having relatively the same pKa, were also active in the assays.

When an assay was completed, a portion of the MEM was diluted and placed with the Vero E6 cells and incubated for 3 days at 37° C. and the plaques were evaluated using an automated counter. It was assumed that each plaque was the result of one active virus particle being responsible for the development of one plaque in the Vero E6 assay system. Each assay was performed in triplicate and the data points were averaged with the standard deviations presented.

Analysis was performed using both gas chromatography/mass spectroscopy (GC/MS) and gas chromatography/flame ionization detection (GC/FID), along with colorimetric titration of the virus wells. Since the initial assays for the potential bioactivity of the Sx were performed in the gas phase, it was important to estimate the quantities of the acid and the esters that were dissolved in the MEM well containing the virus as a guide for concentrations to be tested under other direct injection assay conditions. To this end, the propionic acid component was determined by two methods, including titration of the MEM medium (which already contains phenol red and an acid/base indicator) with a 1 M NaOH solution. The determinations presented were the average of triplicate measurements. The second method for determining the acid content of the MEM was GC/FID, where samples taken from the wells were diluted 20-fold and separated by GC and the amounts measured by FID. The samples were injected into a Hewlett Packard® 6890 gas chromatograph containing a 30 m×0.25 mm inner diameter ZB Wax capillary column with a film thickness of 0.50 mm. A thermal program of 30° C. for 2 min followed by an increase to 220° C. at 5° C./min was applied. Ultrahigh purity helium gas was used as the carrier gas and the initial column head pressure was 50 KPa. The analyses on each sample were also performed in triplicate.

The presence of the esters was determined both qualitatively and quantitatively by GC/MS using a Hewlett Packard® 6890 gas chromatograph. The samples were diluted 2-fold and the initial identification of the esters found in the analysis was made via library comparison using the National Institute of Standards and Technology (NIST) database. In each case (with GC/MS and GC/FID), standardization of the measurements was performed using authentic samples of the propionic acid and the isoamyl hexanoates. All experiments were performed in triplicate. It should be noted that because of the virulent nature of the viruses being tested, all precautions were taken to protect those doing the research, including the extensive use of personal protective equipment (PPE) and other sanitation measures.

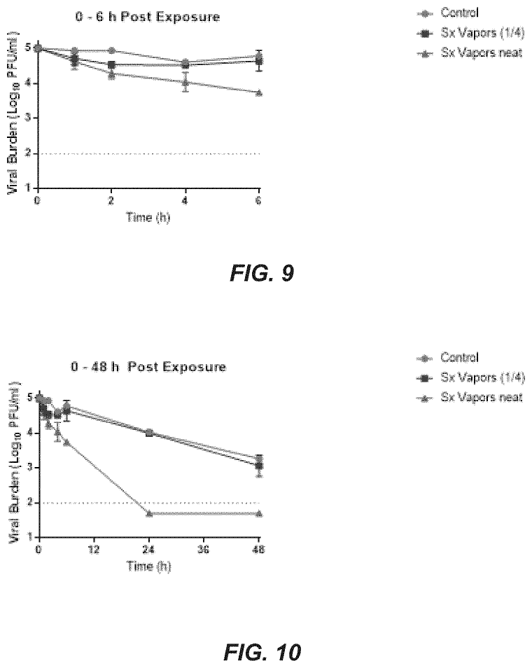

Initially, gas phase testing was conducted with the Sx mixture in the presence of a suspension of virions in the MEM Eagles medium in order to learn the approximate effective concentrations of Sx that might be used in the standard assay, as described above. The gas phase testing seemed ideal, since only small amounts of the Sx in the center well would evaporate and then become dissolved in the MEM buffer well containing the viroids. If any antiviral activity was noted the amount of Sx components in the medium could be estimated by the methods described. In all cases, the control test was one in which no Sx was present in the Petri plate. This technique mirrored that used to ascertain if the mimicked mixture of the Muscodor sp. VOCs possessed antimicrobial activities. At 20° C., there was little to no activity of the Sx, except at 50 μl at 6 hr of exposure (as shown in FIG. 9). This activity was related to between 5.6 and 8.6 μl of propionic acid having become dissolved in the viroid containing well (see Table 3 below). However, up to 24 hr, the virus was gradually 100% inactivated and between 10.7 and 30.8 μl of propionic acid was detected in the viroid well (see FIG. 10 and Table 3 below).

Table 3 below provides an estimation of the amounts of propionic acid and isoamyl hexanoates evaporating from the central well and re-dissolving into the buffered virus suspension as a function of time, concentration and temperature. The amounts of propionic acid were determined both by titration with 1N NaOH as well as GC/FID. The amount of isoamyl hexanoates was estimated by GC/MS. In Table 3, “*” indicates the point at which there was a 100% inactivation of SARS-CoV-2 and “\hat{ }” shows the conditions in which the influenza virus was inactivated. It should be noted that the data obtained from titration determinations and GC/FID are generally in close agreement, but some discrepancies do exist and these are not explainable.

TABLE 3
Estimation of amounts of propionic acid and isoamyl
hexanoates evaporating from the central well and
re-dissolving into the buffered virus suspension
as a function of time, concentration and temperature

| | | | Microliters | Microliters | |
|---|---|---|---|---|---|
| | | | of propionic | via GC/ | |
| | | | acid via | FID ± | |
| | | | titration ± 8% | 0.6-3.7% | Microliters |
| Temper- | Amount | Time | of mean/ | of the mean- | via |
| ature | (μL) | (hr) | triplicate | triplicate+ | GC/MS+ |
| 22 | 25 | 2 | 2.0 | | |
| 22 | 25 | 4 | 4.4 | | |
| 22 | 25 | 6 | 6.6 | 5.9± | 0 |
| 22 | 25\hat{ } | 24\hat{ } | 15.5\hat{ } | 6.84\hat{ } | 0\hat{ } |
| 22 | 50 | 2 | 2.5 | 6.1 | |
| 22 | 50\hat{ } | 4\hat{ } | 5.1\hat{ } | | 0 |
| 22 | 50 | 6 | 8.6 | 5.6 | 0 |
| 22 | 50 | 24* | 30.8* | 10.7* | 0* |
| 37 | 25 | 2 | 2.6 | | |
| 37 | 25 | 4 | 5.5 | 6.4 | 0 |
| 37 | 25\hat{ } | 6\hat{ } | 8.6\hat{ } | | |
| 37 | 25 | 24* | 16.1* | 5.8* | 0 |
| 37 | 50\hat{ } | 1\hat{ } | 1.4\hat{ } | | |
| 37 | 50 | 2 | 7.7 | 6.1 | 0 |
| 37 | 50 | 4* | 16.2* | | 0 |
| 37 | 50 | 6 | 22 | | 0 |
| 37 | 100 | 2 | 9.7* | 22.5* | 1.4* |
| 37 | 100 | 4 | 20.5 | | |
| 37 | 100 | 6 | 25.5 | | |

+Only samples shown were determined and the others were not sampled.
*These samples represented the initial times and concentrations of Sx in which there was compete inactivation of the SARS-CoV-2 virus.
\hat{ }These samples represent the conditions in which the influenza virus was inactivated.

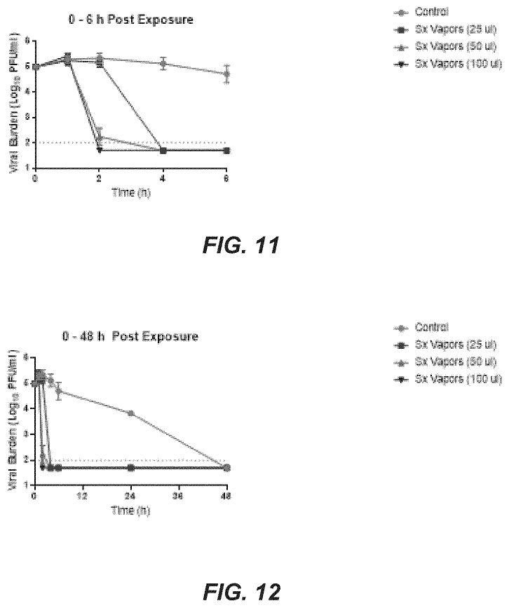

At 37° C. under the 50 μL of Sx in the Petri plate, there was complete inactivation of the SARS-CoV-2 virus at only 2 hr, which equated to between 6.1 and 7.7 μL of propionic acid in the viroid well (see FIG. 11 and Table 3). The nearly identical response to the Sx was observed when 100 μL of Sx was the test mixture, resulting in complete inactivation of the virus in 2 hr at 37° C. (see FIGS. 11 and 12). At this higher amount of test mixture, there was between 22.5 and 9.7 μL of propionic acid found in the viroid well (see FIGS. 11 and 12, and Table 3). Interestingly, it took 4 hr to inactivate the virus with 25 μL of Sx in the center well, and this resulted in 6.4 and 5.5 μL of propionic acid in the viroid well (see FIGS. 11 and 12, and Table 3). Of note was the fact that the isoamyl hexanoates were only detected in the sample exposed for 2 hr to the 100 μL of Sx. This was expected, since there was an elevated temperature and concentration of the esters and the esters are only sparingly soluble in water because of their hydrophobic nature. The action of Sx vapors on Sx in suspension is temperature-dependent, as can be seen in FIGS. 9 and 10 when compared with the data in FIGS. 11 and 12.

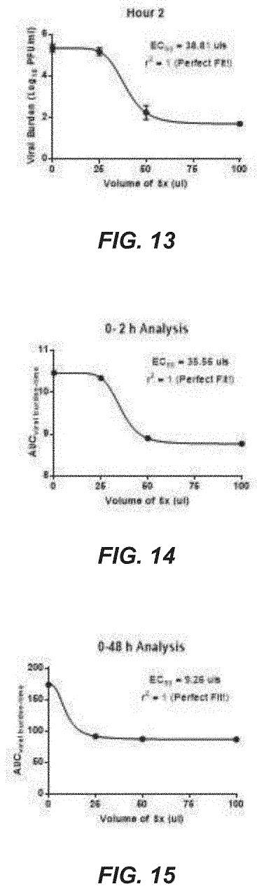

Statistical analysis of the results in FIGS. 13-15 demonstrates a near perfect significant match, indicating the antiviral effects of Sx on SARS-CoV-2. Most interesting, however, was the fact that it took a range of 6 to 8 μL of propionic acid to inactivate the virus at 37° C., but not at the lower temperature (see FIGS. 9 and 10 vs. FIGS. 11 and 12), and for most tests it appeared that the acid alone was the primary active ingredient, since no isoamyl hexanoates were detectable in the MEM viroid containing wells (see Table 3). This effectively allowed us to proceed to the next logical steps in learning if the Sx mixture, when directly injected into the viroid suspension, would result in inactivation of the virus and also what, if any, might be the role of the esters, since they are critical for the antimicrobial activity. Further, central to this work is a working estimate of the range of volumes of Sx that may be used in the direct injection test, and this turned out to be in the low range of 2 μL up to 20 μL of the Sx mixture based on the gas assay tests.

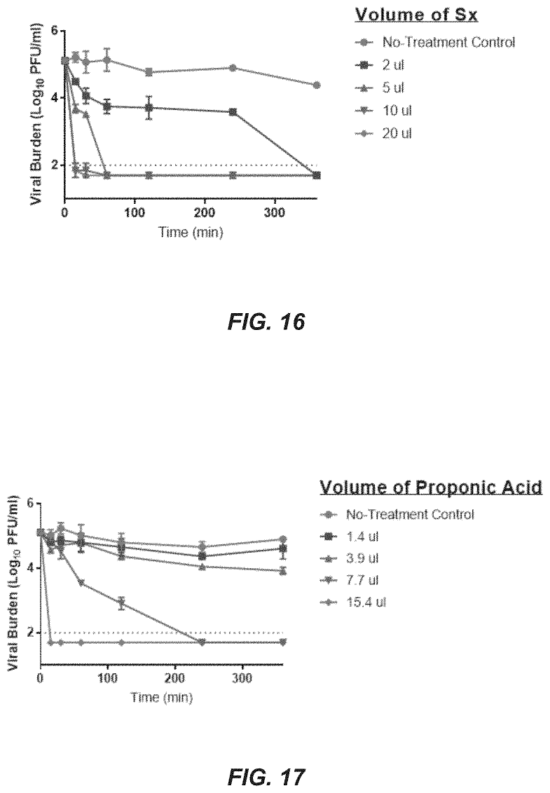

To test direct injection assays of complete Sx on viroid suspensions in the MEM buffer, the MEM buffer (containing 10⁵ virions) was injected and gently stirred with varying amounts of the Sx mixture and incubated at 37° C. Inactivation of the virus occurred at all levels of Sx and at all times, with complete inactivation occurring within 10 min at the 10 and 20 μL levels of the Sx (see FIG. 16). However, at the 2 μL level, over 300 min was required for complete inactivation and about 50 min was required for inactivation at the 5 μL level (see FIG. 16). Clearly, this was much greater antiviral activity than observed in the gas phase testing and probably related to the fact that the optimum ratio of acid to esters was present in the mixture.

Direct injection assays of the Sx in virus suspensions did yield a better assessment of its activity against the virus, since both components of the Sx were present in the MEM buffered virus suspension. The data (see FIG. 16) also showed the unequivocal importance of the isoamyl hexanoates as critical to propionic acid in its antiviral activity, which was initially only noted with the acid alone since neither ester became dissolved in the virus containing well (see FIGS. 9 and 10, and Table 3). Affirmation of the involvement of the individual components in virus inactivation came in experiments in which individual components of the Sx mixture were directly injected into the MEM virus well and then assayed. In these tests, the individual components were tested relative to the proportions that they occupy at the 7:2 v/v ratio of acid to esters. The results of the propionic acid testing alone showed that to get a relative immediate inactivation of the virus, at least 15.4 μL of the acid were necessary, which is comparable to that observed in the gas phase testing (see FIGS. 11, 12 and 17, and Table 3). Additionally, up to 7.7 μL of the acid were necessary to get any appreciable activity (see FIG. 17).

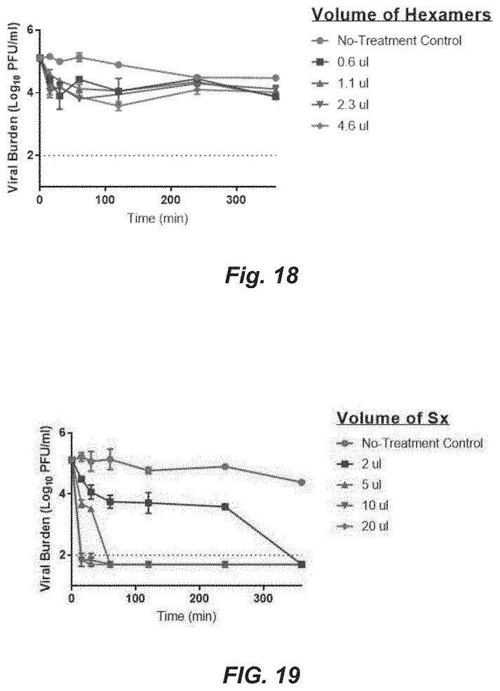

Similarly, when the esters alone were tested, there was a general 1 log reduction in virus activity within about 30 min over the range of concentrations being tested which, after 300 min, about equaled that of the control. Examination of any given point, such as the 5 μL proportionate concentrations of the acid at 3.9 μL and the esters at 1.1 μL, revealed that the individual components demonstrated much less activity than the mixture alone. For example, the acid at 3.9 μL caused only about 0.5 log reduction in activity of the virus, where the esters caused about a 1 log reduction in activity at about 100 min (see FIGS. 17 and 18). However, when the acid and esters (mixture) were directly injected into the virus suspension at the 5 μL level, there was a complete inactivation of the virus in 50 min (4 logs; see FIG. 16). This is exactly what is observed with the anti-microbial activities of Sx. The acid and esters act synergistically to inactivate the virus, which is comparable to what is observed when Sx is tested for its anti-microbial activities.

The Sx mixture was found to be active against SARS-CoV-2 in the gas phase, but most of the activity observed was mainly due to the propionic acid, which evaporated from the chemical well and became dissolved in the MEM buffer. However, direct injections of the Sx mixture into the suspension of viroids resulted in the complete inactivation of the virus at the 10 μL level in less than 10 min (see FIG. 16). This amount of Sx equals 1% of the total volume of liquid and represents the exact amount of Sx in an animal product used in an electrolyte product used to treat diarrhea in animals. Thus, it would appear that the Sx formula may have applications to the human COVID-19 disease, and to treating diarrhea associated with that disease.

The antiviral activity of the Sx formula appears to be the result of propionic acid acting synergistically with the isoamyl hexanoates. It is speculated that the Sx may inactivate the virus by interacting with the receptor binding domain (RBD) of the spike protein which plays an important and critical role in the attachment of the virus to the ACE2 binding site on human cells. It turns out that 10 nm away from the RBD, there is a polybasic cleavage site which is critical for virus infectivity. It appears that flooding this positively charged site (arginine) with negatively charged anions in the presence of a strong lipophilic agent tends to neutralize this critical site and render it ineffective. In fact, this was performed by adding a synthetic peptide GluGluLeuGlu to the virus, reducing its activity by 34 percent. It is to be noted that the leucine was added to the synthetic peptide formula to improve the binding efficiency of the peptide to the ACE2 binding site. In this case, the esters may serve the same purpose.

The activity of the Sx against SARS-CoV-2, as shown above, has the potential for some utility in treating some of the symptoms associated with COVID-19 in the human population. As discussed above, about 20% of patients suffering from COVID-19 develop diarrhea and other gastrointestinal difficulties. Administration of an electrolyte solution containing 1% Sx may alleviate this condition, as at this concentration, the Sx inactivates SARS-CoV-2 in a matter of minutes (see FIG. 16). This suggestion naturally follows the utility of using a 1-2% electrolyte solution in the extremely successful treatment outcome of PED in piglets, which is caused by a coronavirus. In addition, Sx formulated (1%) as a mouth rinse may help prevent the viral load in the upper respiratory system; similarly, Sx may prove helpful in reducing the viral load when administered via an atomizer, nebulizer or intubation directly into the lung tissues. Finally, it seems likely that Sx could also be used conveniently to decontaminate surfaces, instruments, and PPE (personal protective equipment), such as during the course of the COVID-19 pandemic, especially since it is also active in the gas phase (see FIGS. 9-12).

In additional experiments, a direct injection of 2 μL, 5 μL and 10 μL of the Sx mixture into a 1 mL suspension of SARS-CoV-2 particles resulted in, within a time frame of 6 hours, the EC₅₀ was 2.1 μL, with an EC₉₀ of 4.3 μL. Assays performed with propionic acid alone showed complete inactivation of the virus at 7.7 μL in 220 minutes. The esters alone, at the highest concentration of 4.6 μL, showed only a 1 order of magnitude reduction in antiviral activity over the course of 300 minutes.

As a non-limiting example, the Sx mixture may be used for the treatment of new onset symptomatic diarrhea and associated gastrointestinal symptoms in adult patients with COVID-19-particularly that which is documented by PCR detection of SARS-CoV-2 virus—and who do not require mechanical ventilatory support. In addition to the above, the Sx mixture may also be used to treat acute gastroenteritis.

Using standard assay methods, 1.0 mL of a suspension of 10⁵ SARS-CoV-2 virions in MEM 1×Eagles medium were injected with 2 μL, 5 μL, 10 μL and 20 μL of the Sx mixture. At various times, the suspension was assayed for active virions by mixing and plating with a wild Vero E6 human cell line. Plaque counts were made indicating if the Sx mixture was antiviral. Inactivation of the virus occurred at all levels of the Sx mixture and at all times, with complete inactivation occurring within 10 minutes at the 10 μL and 20 μL levels. However, at the 2 μL level, over 300 minutes were required for complete inactivation, and about 50 minutes were required for inactivation at the 5 μL level (see FIG. 19). Table 4 below shows the mass and volume of each component of the overall Sx mixture used for each test.

TABLE 4
Sx Components by Mass and Volume

| Volume (μL) of | Amount added (μg) and final concentration (μg/mL) after addition to |
|---|---|
| Sx test mixture | the 1.0 mL viral suspension |
| 2 μL | 1.4 μg of propionic acid (final concentration ~1.4 μg/mL) and |
| | 0.5 μg of isoamyl hexanoates (final concentration ~0.5 μg/mL) |
| 5 μL | 3.9 μg of propionic acid (final concentration ~3.9 μg/mL) and |
| | 0.98 μg of isoamyl hexanoates (final concentration ~0.98 μg/mL) |
| 10 μL | 7.7 μg of propionic acid (final concentration ~7.7 μg/mL) and |
| | 1.97 μg of isoamyl hexanoates (final concentration ~1.97 μg/mL) |
| 20 μL | 15.4 μg of propionic acid (final concentration ~15.4 μg/mL) and |
| | 3.95 μg of amyl hexanoates (final concentration ~3.95 μg/mL) |

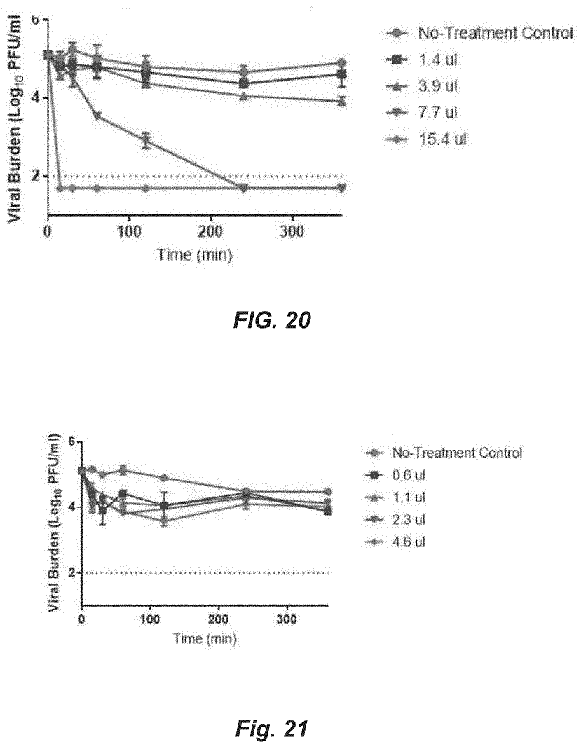

With regard to testing the antiviral activity of propionic acid alone and isoamyl hexanoates alone against SARS-CoV-2, using standard assay methods, the proportional amount of propionic acid was used in the standard assay to learn what contribution this compound was making to inactivation of the virus. The results of the propionic acid testing alone showed that, to acquire a relative immediate inactivation of the virus, at least 15.4 μL of the acid were necessary, and up to 7.7 μL of the acid were necessary to get any appreciable activity, as shown in FIG. 20. Similarly, when the esters alone were tested, there was a general 1 log reduction in virus activity within about 30 minutes over the range of concentrations being tested which, after 300 minutes, approximately equaled that of the control (see FIG. 21). Upon examination of any given point, such as the 5 μL proportionate concentrations of the acid at 3.9 μL and the esters at 1.1 μL, it was revealed that the individual components demonstrated much lower activity than the mixture alone. For example, the acid at 3.9 μL caused only about 0.5 log reduction in activity of the virus, and the esters caused about a 1 log reduction in activity at about 100 minutes, as shown in FIGS. 20 and 21. However, when the mixture of the acid and esters was directly injected into the virus suspension at the 5 μL level, there was a complete inactivation of the virus within 50 minutes.

Although it appears that the main active compound in the Sx mixture is propionic acid, alone, it only has marginal biological activity. However, when combined with the isoamyl hexanoates, the activity of the mixture is greatly enhanced, as can be seen by comparing FIGS. 20 and 21 against FIG. 19. Since the isoamyl hexanoates have no lasting activity in the assay, exceeding that of the control alone at 300 minutes (see FIG. 21), these compounds cannot be considered to have any substantial anti-viral activity. Thus, they may be classified as potentiators or enhancers of the activity of the propionic acid. In immunological terms, they act as adjuvants to increase antigen production by a biological system.

It should also be noted that the activity level of the Sx mixture at 5 to 10 μL per mL is at 0.5% to 1% of the final MEM buffer solution, and this is within an ideal range for administration to a patient. Additionally, it should be noted that the ingredients are GRAS listed and are well below the range of any noted toxicity. It should be understood that, as used herein, the term “patient” may refer to either a human or animal patient.

For the above-described experiments, SARS-CoV-2 virus was obtained from BEI resources (NR-52282 SARS-Related Coronavirus 2 Isolate Hong Kong/VM20001061/2020) and the wild Vero E6 cells were obtained from ATCC, Manassas, VA. Propionic acid and the isoamyl hexanoates were obtained from Eccelentia International Co., Fairfield N.J. Other chemicals were obtained from Sigma/Aldrich Chem Co. In each experiment, as discussed above, a 1.0 mL volume of a suspension of 10⁵ SARS-CoV-2 virions in MEM 1×Eagles medium in individual wells was injected with the test solution. A series of sequential μL volumes of the test solution were tested in separate wells of the viral suspension. Antiviral activity was assessed at sequential prespecified time intervals following injection of the test solution into the 1 mL viral MEM suspension. The suspension was assayed for active virions by mixing and plating with a wild Vero E6 human cell line. At sequential time intervals after injection of the test solution, a portion of the MEM viral suspension was diluted and placed with the Vero E6 cells and incubated for 3 days at 37° C. The number of plaques were evaluated using an automated counter. It was assumed that each plaque was the result of one active virus particle being responsible for the development of one plaque in the Vero E6 assay system. Each assay was performed in triplicate and the data points averaged and the standard deviations presented. All experiments were carried out in virology laboratory facilities, including the use of personal protective equipment, appropriate for handling of live SARS-CoV-2 virus.

As discussed above, the Sx mixture may be administered to a patient using any suitable method. Table 5 below provides a non-limiting example of an oral formulation of the Sx product.

TABLE 5
Formulation of Orally Administered Sx Product

| Component | Final Concentration | |
|---|---|---|
| Propionic acid | 7.7 | mg/mL |
| Isoamyl hexanoates** | 2.0 | mg/mL |
| ** 99% 3-methyl butyl hexanoate and | | |
| 1% 2-methyl butyl hexanoate | | |
| Potassium chloride | 5.0 | mg/mL |
| Magnesium chloride heptihydrate | 5.0 | mg/mL |
| Mono sodium phosphate | 2.5 | mg/mL |
| Sodium citrate | 5.0 | mg/mL |
| Dextrose monohydrate | 40.0 | mg/mL |
| Sucralose | 83.3 | μg/mL |

| | |
|---|---|
| Cherry liquid flavoring | ~1% total volume |
| RO/DI water for pharmaceutical use | QS to final volume of 1 mL |

The orally administered formulation described in Table 5 is an aqueous solution which, for patients with diarrhea, has a proposed dosing schedule of 50 mL twice daily (˜0.8 mL per kg for 60-65 kg adult) for five (5) days. For a proposed 1 st-in-human phase 1 study, the oral formulation may be administered using randomized subjects for receiving the treatment solution vs. subjects receiving a placebo solution in a double-blind protocol. Based on current literature regarding new onset diarrhea as the presenting symptom in adults with COVID-19, a five (5) day treatment period may be sufficiently long to detect a signal of potential efficacy in reduction in frequency and severity of diarrhea in the active treatment vs. the placebo cohort. In addition, in contrast with the prolonged period of viral RNA shedding in the gastrointestinal tract, recovery of viable SARS-CoV-2 virus from gastrointestinal stool samples appears to be limited to a few day period after onset of symptoms.

Twice daily dosing, rather than one-time or once daily dosing, is contemplated because viable SARS-CoV-2 virus is currently understood to infect intestinal lining cells via abundant ACE2 with subsequent intracellular production and extracellular release of newly formed virions. Thus, “re-inoculation” of the GI tract with viable SARS-CoV-2 virus may be better treated using repeated daily dosing rather than one-time dosing or once daily dosing.

Additional potential uses of the combination of propionic acid and/or isobutyric acid, along with the isoamyl hexanoates, includes without limitation: deodorizing cat litter and other waste locations or sites; treatment or prevention of diarrhea generally; treatment or relief from symptoms of viral infections generally; or treatment of acute gastroenteritis generally. Generally, compositions for any of these treatments that include both propionic and isobutyric acids include these two components at a 50:50 v/v proportion. In combination with isoamyl hexanoates, the relative proportions frequently are 3.5:3.5:2 v/v/v, acid:acid:ester.

The Sx composition has also been found to be effective against parasitic infections, such as Cryptosporidium, coccidia, and giardia. 36 Holstein-Friesian bull and heifer calves, each between two to three weeks old at the time of enrollment, were housed in individual calf hutches and exposed to ambient environmental conditions. The calves were divided into three groups: a group fed a supplement of the Sx composition (hereinafter referred to as “Sx Calf”), a group fed a supplement with a concentrated form of the Sx composition (hereinafter referred to as “Sx Calf Concentrate”), and a control group, who ingested untreated saline. All animals enrolled in the study exhibited diarrhea and had been diagnosed with Cryptosporidium based on ELISA testing of fecal samples by the California Animal Health and Food Safety Laboratory located in Tulare, CA.

The control group received untreated saline as a single oral drench at a volume of 50 ml per calf on Day 0. Sx Calf was administered as an oral drench at a volume of 50 ml per calf on Day 0. Calves treated with Sx Calf were observed approximately 8 hours after dosing to evaluate their hydration status. Calves that still appeared to be dehydrated were dosed with an additional 50 ml of Sx Calf by oral drench. Only one calf required a second dose. Sx Calf Concentrate was administered as an oral drench at a volume of 0.067 ml per pound twice per day approximately 8 hours apart on Days 0, 1 and 2. The control saline and Calf Sx were administered orally using a 60 ml catheter tipped syringe. Calf Sx Concentrate was administered with a 10 ml syringe. Care was taken to ensure the calves did not aspirate the materials. Sx Calf and Sx Calf Concentrate were shaken before administration to ensure a homogeneous suspension was achieved.

Fecal samples were collected on Day 3 and Day 7. Samples were submitted to the California Animal Health and Food Safety laboratory for analyses, including ELISA for Cryptosporidium oocysts and rotavirus. Microbiological analyses to identify the presence of Salmonella sp. were also performed. Results of analyses were documented on the standard report forms used by the laboratory. Calves were observed twice daily beginning on Day 0 through Day 7. Illness Scores ranging from normal to moribund were recorded using a visual analog scale. Fecal scores were assigned for each calf once per day using a scale of 0-3, where 0=normal consistency, 1=semiformed or pasty consistency, 2=loose feces, or 3=watery feces.

No mortalities occurred among animals enrolled in the study. In addition, no animals were removed from the study following enrollment due to positive Salmonella sp. culture results. Any abnormal health observation that was unexpected within the scope of the study objectives was defined as a suspected adverse drug experience. There were no adverse drug experiences observed during the course of the study. Table 6 summarizes the proportion of animals in each treatment group exhibiting abnormal feces. 100% of calves in all treatment groups exhibited mild to moderate diarrhea prior to treatment.

TABLE 6
Comparative Results

| | Day | Day | Day | Day | Day | Day | Day | Day |
|---|---|---|---|---|---|---|---|---|
| | 0 | 1 | 2 | 3 | 4 | 5 | 6 | 7 |
| Control | 12/12 | 12/12 | 10/12 | 10/12 | 6/12 | 7/12 | 8/12 | 7/12 |
| Saline | | | | | | | | |
| Sx Calf | 12/12 | 9/12 | 5/12^{a} | 2/12^{c} | 1/12^{a} | 1/12^{b} | 1/12^{c} | 0/12^{c} |
| Sx Calf | 12/12 | 8/12^{a} | 2/12^{c} | 0/12^{c} | 0/12^{b} | 0/12^{c} | 0/12^{c} | 0/12^{c} |
| Conc. | | | | | | | | |

^{a}significantly different from saline controls p < 0.1
^{b}significantly different from saline controls p < 0.0.05
^{c}significantly different from saline controls p < 0.01

As can be seen in Table 6 above, administration of Sx Calf significantly reduced the proportion of calves with abnormal feces relative to saline controls beginning two days after initiating treatment and completely eliminated abnormal feces by seven days after treatment started. Administration of Sx Calf Concentrate significantly reduced the proportion of calves with abnormal feces relative to saline controls beginning the day after dosing was started, and completely eliminated abnormal feces by three days after treatment started. There were no statistically significant differences between the responses of calves to Sx Calf and Sx Calf Concentrate. However, it appeared that the improvement in fecal scores was more rapid for calves treated with Sx Calf Concentrate relative to calves treated with SX Calf.

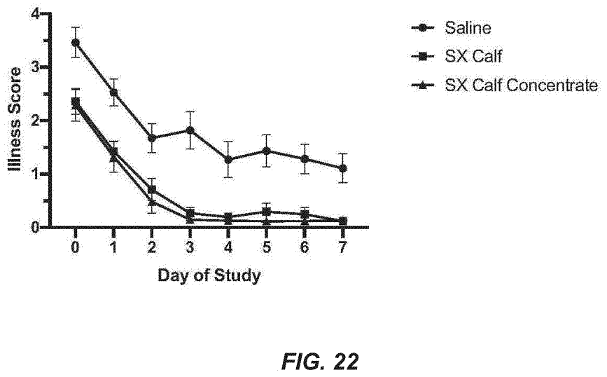

The mean daily illness scores for animals in each treatment group are summarized in FIG. 22. Animals treated with either Sx Calf or Sx Calf Concentrate exhibited a decrease in illness scores relative to the saline control calves following initiation of treatment. Calves treated with Sx Calf and Sx Calf Concentrate returned to near normal appearance on Days 3-7 following initiation of treatment, whereas calves in the saline control group exhibited moderate to mild illness scores throughout the duration of the study. The observed reductions in illness scores relative to the saline controls were statistically significant (p<0.01).

The results of the laboratory analyses on fecal samples collected from the calves prior to enrollment and on Day 7, following the initiation of treatment, confirmed that none of the animals enrolled in the study had Salmonella in their feces at either of the sampling timepoints. Table 7 summarizes the incidence of Cryptosporidium and rotavirus positive fecal samples per treatment group prior to treatment and on Day 7.

TABLE 7
Incidence of Cryptosporidium and rotavirus

| | |
|---|---|
| Pre | Day 7 |

| Treatment | Crypto (+) | Rota (+) | Crypto (+) | Rota (+) |
|---|---|---|---|---|
| Saline | 12 | 0 | 8 | 1 |
| Sx Calf | 12 | 0 | 8 | 6^{a} |
| Sx Calf Conc. | 12 | 0 | 9 | 3 |

^{a}statistically different from saline controls (p < 0.1)

Prior to the initiation of dosing, all calves in all three treatment groups were positive for Cryptosporidium and negative for rotavirus. On Day 7, approximately 65-75% of the calves in each treatment group remained positive for Cryptosporidium in their feces. There were no statistically significant (p>0.5) differences in the incidence of Cryptosporidium positive fecal samples on Day 7 between treatment groups. On Day 7, 8% of the negative control calves tested positive for rotavirus in their fecal samples. In contrast, 50% of the Sx Calf treated calves and 25% of the Sx Calf Concentrate treated calves tested positive for rotavirus on Day 7. The incidence of rotavirus positive samples was significantly (p<0.1) greater in calves treated with Sx Calf than the saline controls. The difference in incidence of rotavirus positive fecal samples on Day 7 between Sx Calf and Sx Calf Concentrate treated calves was not statistically significant.

These results suggest the reductions in the incidence of diarrhea observed among animals treated with either Sx Calf or Sx Calf Concentrate were not due to elimination of Cryptosporidium from the GI tract of the animals. Since identification of Cryptosporidium in this study was determined using an ELISA assay to detect oocyst antigens in fecal samples, it is not possible to determine if the Cryptosporidium present on Day 7 were viable. The presence of rotavirus on Day 7 in calves which did not test positive prior to treatment indicates the product does not inhibit subsequent infection with viral pathogens associated with diarrhea in calves.

### Animal Feed Compositions and Use

The animal feed composition can be prepared as a single- or multi-species animal feed made from a base animal feed, a carrier, and a supplement carried by the carrier. The supplement, if using the Sx product, includes propanoic acid and isoamyl hexanoates. Other effective variations of the formula are available as set forth above. For use of the Sx product, the volume ratio of the propanoic acid to the isoamyl hexanoates is preferable 7:2. The concentration of the Sx supplement in the animal feed may be between approximately 0.125% and approximately 0.375% by weight. The supplement in animal feed may be used to improve gut health, to promote weight gain, as an immune modulator, and as a feed preservative. The Sx product and other variations provide these benefits without being an antibiotic. The Sx product or similar variation has antimicrobial, antifungal and antiviral properties, thus promoting gut health and a boost in immunity in animals (when fed orally), such as in monogastric animals, ruminants and poultry, as well as companion pet animals, through action on the animal microbiome. Additionally, the Sx or similar product preserves the feedstuff in storage by inhibiting pathogens, which can cause mold, spoilage, and mycotoxin exposure.

As a non-limiting example, a cattle feed may be prepared, as described above, with a 20% beef starter pellet used as the base animal feed. As a non-limiting example, such a dry cattle feed may include not less than 20% crude protein, 1.5-3.0% crude fat, 10-15% crude fiber, 0.5-1.0% calcium, not less than 0.5% phosphorous, 0.5-1.0% salt, 0.5-1.0% potassium, not less than 20 ppm copper, not less than 0.1 ppm selenium, not less than 150 ppm zinc, not less than 5,000 IU/lb vitamin A, not less than 250 IU/lb vitamin D, and not less than 100 IU/lb vitamin E. As a non-limiting example, the animal feed composition may be fed at a daily rate of 4.0 pounds per head, per day in combination with good quality grass hay or other forage. It should be understood that the animal feed composition may be prepared for use to feed any suitable type of animal, such as, but not limited to, swine, poultry, horses, goats, sheep, llamas and alpacas.

A study on the animal feed composition was conducted at Weddel Ranch in Kamiah, Idaho during May of 2016. In the study, 15 calves were fed the Sx product, in their concentrated feed, in the form of a top dress product. This consisted of a zeolite carrier treated with the Sx product and added to the feed. The calves were fed for 75 days, and the calf weights were obtained both prior to feeding and post feeding. The average weight gain in the calves over 75 days was 1.49 pounds/day. The feed consisted of a native grass hay pellet, top dressed with the Sx product, and free choice prairie grass hay of limited nutritional quality.

### Waste Treatment Compositions and Use

The composition may be applied to human or animal waste to decontaminate, degrade and/or deodorize the waste. As non-limiting examples, the Sx or similar mixture may be used for the treatment of waste in latrines; treatment of bedding such as in animal stalls, barns, chicken-raising facilities, pig barns, pet stations in homes, and zoos; and treatment of litter such as cat litter, or treatment of litter containers such as cat litter boxes. One example is to administer Sx or a similar mixture to zeolite, for example at a rate of about 3 lb of material per 400 square feet of coverage in a stall, or 90 ml of Sx per 3 lbs of zeolite.

As a further non-limiting example, the Sx or similar mixture may be incorporated into poultry litter to control ammonia levels in poultry production facilities. By reducing ammonia levels in brooder pens, the birds are expected to exhibit both weight gain and feed efficiency.

In order to test the Sx mixture for this purpose, it is proposed to test the Sx mixture on 315 one-day old (at the start of the testing) Cobb500 broilers. The broilers will be individually identified with plastic neck tags and will be housed in floor pens within three different rooms in the poultry building. Each room will contain three floor pens, with one treatment per room. Each floor pen will measure approximately 5′×7.5′, providing approximately 1 square foot of surface area per bird. Used rice hull bedding, approximately 3-4″ deep, will be used in each pen.

Temperatures and humidity will be monitored using electronic meters placed in each floor pen. The initial temperature in each floor pen will be maintained at approximately 85° F. using heat lamps and a ventilation system. Temperatures will be reduced approximately 0.5° F. per day until the temperature reaches approximately 75° F. Humidity will be maintained in each room at approximately 50-70% using the heat lamps and ventilation system. The birds will be fed a commercial ration and will be provided with water according to facility standard operating procedures.

Further, no concomitant medications will be used during the duration of the study. All birds to be enrolled in the study will be free of clinically observable abnormalities. Birds exhibiting clinically apparent abnormalities prior to the test will be excluded from enrollment in the study. The study is proposed to be a completely randomized trial, with 10 5 birds being a “negative control” with no litter treatment, 10 5 birds being a “positive control” with a commercially available poultry litter treatment (PLT®-Poultry Litter Treatment, manufactured by the Jones-Hamilton Co.), and 10 5 birds using the Sx mixture-treated poultry litter.

During the testing, the litter in negative control floor pens will not be treated with any product. The positive control floor pens will be treated with the PLT®-Poultry Litter Treatment by applying 3.5 lbs. of the product to the surface of the litter in each pen using a handheld broadcast spreader. The positive control product should not be incorporated into the litter. The Sx mixture-treated poultry litter floor pens will be treated with the Sx mixture-treated poultry litter by applying 0.2 lbs. of the Sx mixture-treated poultry litter to the surface of the litter in each pen using a handheld broadcast spreader. The Sx mixture-treated poultry litter also will not be incorporated into the litter. Both the positive control product and the Sx mixture-treated poultry litter will be applied to the litter approximately 24 hours prior to placing the broilers in the pens.

During testing, temperatures at bird level in each floor pen will be monitored with electronic thermometers. Temperatures will be recorded at 8 AM, 12 PM, 4 PM and 8 PM daily on days 1-14. Humidity at bird level in each floor pen will be monitored with electronic meters. Humidity also will be recorded at 8 AM, 12 PM, 4 PM and 8 PM daily on days 1-14. Ammonia levels at bird level in each floor pen will be monitored with electronic meters. Ammonia levels also will be recorded at 8 AM, 12 PM, 4 PM and 8 PM daily on days 1-14. Daily pen feed consumption will be based upon the difference in feed offered and feed weighbacks recorded on days 1-14. Individual bird bodyweights will be recorded on days 1 and 14 to assess weight gain over the course of the study.

The primary variable that will be used to determine efficacy of the Sx mixture-treated poultry litter will be the ammonia levels in the Sx mixture-treated pens vs. the negative and positive controls. Mean daily humidity levels and ammonia levels per pen will be analyzed using repeated measures analysis of variance. Differences between treatment groups in weight gain and feed efficiency over the 14-day duration of the study will be analyzed by Student's T-test. Differences in the incidence of mortalities per treatment group will be analyzed using Fisher's Exact test. Analyses will be performed using Graphpad Prism 8™ software on a Macintosh® computer. Given the small number of animals involved, the level of significance is set to α=0.1 for the statistical analysis of all parameters.

It is to be understood that the compositions and methods for treating infections and diarrhea associated with infections, for food and food supplements, and for treating human and animal waste, are not limited to the specific embodiments described above but encompass any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A method of treating an infection or treating diarrhea associated with an infection, comprising the step of administering an effective amount of a composition comprising:
isoamyl hexanoates; and
at least one acid selected from the group consisting of lactic acid, propanoic acid, isobutyric acid, butyric acid, formic acid, acetic acid, citric acid, oxalic acid, uric acid, malic acid, tartaric acid, and combinations thereof.

2. The method of treating an infection or treating diarrhea associated with an infection as recited in claim 1, wherein the step of administering the effective amount of the composition to the patient comprises administering the effective amount of the composition to a patient suffering from a condition selected from the group consisting of a fungal infection, infection with protozoa, coronavirus disease 2019 (COVID-19), mastitis, rain rot, scald, foot rot, an ear infection, ring worm, dermatitis, a bacterial infection, a parasitic infection, a viral infection, diarrhea, a Streptomyces avermitilis infection, vaginitis, an oral infection, a nasal infection, a throat infection, jock itch, vaginosis, a toenail infection, a fingernail infection, an eye infection, and combinations thereof.

3. The method of treating an infection or treating diarrhea associated with an infection as recited in claim 1, wherein the step of administering the effective amount of the composition to the patient comprises administering the effective amount of the composition in a gaseous phase.

4. A method of deactivating a virus, comprising the step of administering an effective amount of an antiviral mixture to a patient, wherein the antiviral mixture comprises:
isoamyl hexanoates; and
at least one acid selected from the group consisting of lactic acid, propanoic acid, isobutyric acid, butyric acid, formic acid, acetic acid, citric acid, oxalic acid, uric acid, malic acid, tartaric acid, and combinations thereof.

5. The method of deactivating a virus as recited in claim 4, wherein the virus comprises a SARS-CoV-2 virus.

6. The method of treating SARS-CoV-2 infection as recited in claim 5, wherein the at least one acid includes propanoic acid, and wherein a v/v ratio of the propionic acid to the isoamyl hexanoates in the antiviral mixture is 7:2.

7. The method of deactivating a virus of claim 4, wherein the composition is administered in a gas phase to the patient.

8. The method of deactivating a virus of claim 5, wherein the composition is administered in a gas phase to the patient.

9. The method of deactivating a virus of claim 6, wherein the composition is administered in a gas phase to the patient.

10. A method of promoting gut health, boosting immunity, or preserving feedstuff in storage, the method comprising:
providing a base animal feed,
treating the base animal feed with a supplement comprising isoamyl hexanoates, and
at least one acid selected from the group consisting of lactic acid, propanoic acid, isobutyric acid, butyric acid, formic acid, acetic acid, citric acid, oxalic acid, uric acid, malic acid, tartaric acid, and combinations thereof to produce a feedstuff; and
administering the feedstuff to an animal or storing the feedstuff.

11. The method of promoting gut health, boosting immunity, or preserving feedstuff in storage of claim 10, wherein the supplement is administered in a gas phase to the base animal feed.

12. A method of waste treatment, the method comprising the steps of
preparing a composition comprising isoamyl hexanoates; and at least one acid selected from the group consisting of lactic acid, propanoic acid, isobutyric acid, butyric acid, formic acid, acetic acid, citric acid, oxalic acid, uric acid, malic acid, tartaric acid, and combinations thereof; and
treating waste with the composition.

13. The method of waste treatment of claim 12, wherein the step of treating the waste comprises one or more of the following: decontaminating, degrading, and deodorizing the waste.

14. The method of waste treatment of claim 12, wherein the step of treating the waste comprises administering the composition to one or more of the following: waste, litter, and bedding.

15. The method of waste treatment of claim 13, wherein the step of treating the waste comprises administering the composition to one or more of the following: waste, litter, and bedding.

16. The method of waste treatment of claim 12, wherein the waste is treated with the composition in a gas phase.

17. The method of waste treatment of claim 13, wherein the waste is treated with the composition in a gas phase.

18. The method of waste treatment of claim 14, wherein the waste is treated with the composition in a gas phase.

19. The method of waste treatment of claim 15, wherein the waste is treated with the composition in a gas phase.
